(19) United States
(12) Patent Application Publication
(10) Pub. No.: US 2023/0218892 A1
(43) Pub. Date: Jul. 13, 2023

(54) METHOD AND APPARATUS FOR TREATING SLEEP APNEA

(71) Applicant: Invicta Medical, Inc., Santa Clara, CA (US)

(72) Inventors: Walter Joseph Stevens, San Jose, CA (US); David Herron, Los Angeles, CA (US); Chang Yeul Lee, San Jose, CA (US); Ling-Kang Tong, Fremont, CA (US); Paul Thomas Hichwa, Mountain View, CA (US); Hoa D. Nguyen, San Jose, CA (US); Nishant Srivastava, Santa Clara, CA (US); Eymard Julio Burlaza, Lathrop, CA (US)

(21) Appl. No.: 18/087,595

(22) Filed: Dec. 22, 2022

Related U.S. Application Data
(62) Division of application No. 16/811,998, filed on Mar. 6, 2020, now abandoned.
(60) Provisional application No. 62/814,686, filed on Mar. 6, 2019.
(60) Provisional application No. 62/916,162, filed on Oct. 16, 2019.

(51) Int. Cl. A61N 1/05; A61N 1/36

(52) U.S. Cl. CPC A61N 1/0548; A61N 1/3611; A61N 1/36139

(57) ABSTRACT

Intraoral appliances are disclosed that provide electrical stimulation to tissue in a patient's oral cavity in a manner that reduces apnea events during sleep. A representative appliance can induce a current or currents through tissue and/or anatomical structures in a manner that maintains upper airway tone and/or patency.

### CROSS-REFERENCE TO RELATED APPLICATIONS

The present application claims priority to the following pending U.S. Provisional Patent Applications: U.S. 62/814,686 (filed Mar. 6, 2019) and U.S. 62/916,162 (filed Oct. 16, 2019), both of which are incorporated herein by reference.

### TECHNICAL FIELD

Representative devices and methods disclosed herein relate generally to treating obstructive sleep apnea, and in particular embodiments, to non-invasive methods and devices for treating obstructive sleep apnea.

### BACKGROUND

Obstructive sleep apnea (OSA) is a medical condition in which a patient's upper airway is occluded (partially or fully) during sleep, causing sleep arousal. Repeated occlusions of the upper airway may cause sleep fragmentation, which in turn may result in sleep deprivation, daytime tiredness, and/or malaise. More serious instances of OSA may increase the patient's risk for stroke, cardiac arrhythmias, high blood pressure, and/or other disorders.

OSA may be characterized by the tendency of soft tissues of the upper airway to collapse during sleep, thereby occluding the upper airway. OSA is typically caused by the collapse of the patient's soft palate and/or by the collapse of the patient's tongue (typically onto the back of the pharynx or into the upper airway), which in turn may obstruct normal breathing and/or cause arousal from sleep.

Some treatments have been available for OSA including, for example, surgery, constant positive airway pressure (CPAP) machines, and electrically stimulating muscles or related nerves associated with the upper airway to move the tongue (or other upper airway tissue). Surgical techniques have included tracheotomies, procedures to remove portions of a patient's tongue and/or soft palate, and other procedures that seek to prevent collapse of the tongue into the back of the pharynx. These surgical techniques are very invasive. CPAP machines seek to maintain upper airway patency by applying positive air pressure at the patient's nose and mouth. However, these machines are uncomfortable, cumbersome, and may have low compliance rates.

Some electrical stimulation techniques seek to prevent collapse of the tongue into the back of the pharynx by causing the tongue to protrude forward (e.g., in an anterior direction) during sleep. For example, U.S. Pat. No. 4,830,008 discloses an invasive technique in which electrodes are surgically implanted into a patient at locations on or near nerves that stimulate the genioglossus muscle to move the tongue forward (e.g., away from the back of the pharynx). U.S. Pat. Nos. 5,190,053 and 6,212,435 disclose electrically stimulating the genioglossus muscle to move the tongue forward in an anterior direction during apnea episodes. In another example, U.S. Pat. No. 7,711,438 discloses a non-invasive technique in which electrodes, mounted on an intraoral device, electrically stimulate the genioglossus muscle to cause the tongue to move forward during respiratory inspiration. In addition, U.S. Pat. No. 8,359,108 teaches an intraoral device that applies electrical stimulation to the hypoglossal nerve to contract the genioglossus muscle, which, as mentioned above, may prevent upper airway collapse by moving the tongue forward during sleep.

Existing techniques for electrically stimulating the hypoglossal nerve and/or the genioglossus muscle may cause discomfort, sleep arousal and/or pain, which is not desirable. Further, invasive techniques for electrically stimulating the hypoglossal nerve and/or the genioglossus muscle undesirably require surgery.

Thus, there is a need for an improved non-invasive treatment for OSA and other sleep disorders.

### BRIEF DESCRIPTION OF THE DRAWINGS

Representative embodiments are illustrated by way of example and are not intended to be limited by the Figures, where like reference numerals generally refer to corresponding parts throughout.

FIG. 1 is an elevated perspective view of an oral appliance configured in accordance with representative embodiments of the present technology.

FIG. 2 is a side view of the oral appliance of FIG. 1.

FIG. 3 is a bottom plan view of the oral appliance of FIG. 1.

FIG. 4 is an elevated perspective view of an oral appliance configured in accordance with representative embodiments of the present technology.

FIG. 5 is a side view of the oral appliance of FIG. 4.

FIG. 6 is a bottom plan view of the oral appliance of FIG. 4.

FIG. 7 is an elevated perspective view of an oral appliance configured in accordance with representative embodiments of the present technology.

FIG. 8 is a side view of the oral appliance of FIG. 7.

FIG. 9 is a bottom plan view of the oral appliance of FIG. 7.

FIG. 10 is an elevated perspective view of an oral appliance configured in accordance with representative embodiments of the present technology.

FIG. 11 is a side view of the oral appliance of FIG. 10.

FIG. 12 is a bottom plan view of the oral appliance of FIG. 10.

FIG. 13 is an elevated perspective view of an oral appliance configured in accordance with representative embodiments of the present technology.

FIG. 14 is a side view of the oral appliance of FIG. 13.

FIG. 15 is a bottom plan view of the oral appliance of FIG. 13.

FIG. 16 is an elevated perspective view of an oral appliance configured in accordance with representative embodiments of the present technology.

FIG. 17 is a side view of the oral appliance of FIG. 16.

FIG. 18 is a bottom plan view of the oral appliance of FIG. 16.

FIG. 19 is a partially schematic cut-away sagittal view of the patient's head depicting the oral appliance of FIG. 16 positioned in the oral cavity and configured in accordance with representative embodiments of the present technology.

FIG. 20 is an elevated perspective view of an oral appliance configured in accordance with representative embodiments of the present technology.

FIG. 21 an elevated perspective view of an oral appliance configured in accordance with representative embodiments of the present technology.

FIG. 22 is an elevated side perspective view of an oral appliance configured in accordance with representative embodiments of the present technology.

FIG. 23A is an elevated side perspective view of an oral appliance configured in accordance with representative embodiments of the present technology.

FIG. 23B is a partially schematic cut-away sagittal view of the patient's head depicting the oral appliance of FIG. 23A positioned in the oral cavity and configured in accordance with representative embodiments of the present technology.

FIGS. 23C and 23D illustrate representative external electrodes configured to provide electrical current to a patient in accordance with representative embodiments of the present technology.

FIG. 24 is an elevated perspective view of an oral appliance configured in accordance with representative embodiments of the present technology.

FIG. 25 is a side perspective view of the oral appliance of FIG. 24.

FIG. 26 is a bottom plan view of the oral appliance of FIG. 24.

FIG. 27 is a side view of an electrode array of an oral appliance configured in accordance with representative embodiments of the present technology.

FIG. 28A is a partially schematic, cut-away coronal view of a patient's head depicting electrodes positioned in the oral cavity and configured in accordance with representative embodiments of the present technology.

FIG. 28B is a partially schematic, cut-away sagittal view of the patient's head depicting electrodes positioned in the oral cavity and configured in accordance with representative embodiments of the present technology.

FIG. 28C is a partially schematic, cut-away transverse view of the patient's head depicting electrodes positioned in the oral cavity and configured in accordance with representative embodiments of the present technology.

FIG. 29A is a side sectional view depicting a patient's upper airway.

FIG. 29B is a front plan view of the patient's oral cavity.

FIG. 29C is an elevated sectional view of the patient's tongue.

FIG. 29D is a side sectional view of the patient's tongue.

FIG. 30A is a side sectional view of a patient's tongue with an electrode of an intraoral stimulation device in an optional position for stimulation, in accordance with representative embodiments of the present technology.

FIG. 30B is a frontal section anterior view sectioned behind the first molar of the patient's oral cavity and with an electrode of an intraoral stimulation device in an optional position for stimulation, in accordance with representative embodiments of the present technology.

FIG. 31A is a representative example of a stimulation cycle of a stimulation waveform.

FIG. 31B is a representative example of a stimulation waveform including active and resting periods.

FIG. 32A is a front elevational view of an intraoral appliance configured in accordance with representative embodiments of the present technology.

FIG. 32B is a back elevational view of an intraoral appliance configured in accordance with representative embodiments of the present technology.

FIG. 33A is an elevated perspective view of an extension member configured in accordance with representative embodiments of the present technology.

FIG. 33B is a side view of the extension member of FIG. 33A.

FIG. 33C is a bottom plan view of an oral appliance with the extension members of FIGS. 33A and B.

### DETAILED DESCRIPTION

1. Introduction

Electrostimulation treatments for obstructive sleep apnea (OSA) typically involve modulating or stimulating nerves and/or muscles, e.g., to cause the tongue or other soft tissue to move in order to remove an obstruction of the upper airway, or to prevent the tongue or other soft tissue from collapsing or obstructing the airway. As used herein, the terms “modulate” and “stimulate” are used interchangeably to mean having an effect on, e.g., an excitatory effect, inhibitory effect, and/or other effect. Such stimulation may be provided to one or more nerve branches or muscles of the upper airway structures.

Representative methods and apparatuses for reducing the occurrence and/or severity of a breathing disorder, such as OSA are disclosed herein. In accordance with representative embodiments, a non-invasive and removable oral appliance provides electrical stimulation to anatomical structures of a patient's oral cavity (mouth) in a manner that improves upper airway patency and/or improves the tone of the tissue of the intraoral cavity to treat sleep apnea. An electric current induced by the appliance can stimulate at least a portion of a patient's hypoglossal nerve, genioglossus muscle and/or other nerves or muscles associated with the upper airway. By moving the tongue forward and/or by preventing a collapse of the soft tissue and/or tongue onto the back of the patient's pharynx, and/or into the upper airway, the patency or tone of the patient's upper airway can be improved in a non-invasive manner.

Non-invasive methods and apparatuses for treating a patient, for example, for sleep disorders such as OSA and/or snoring, are disclosed herein. A removeable mouthpiece or oral appliance is disclosed that is secured in the oral cavity and comprises one or more electrodes (e.g., stimulation electrodes). The electrode(s) can be activated to direct electrical current through tissue in the oral cavity to stimulate nerve branches and/or muscles to improve upper airway patency or tone and/or reduce an upper airway obstruction that may contribute to sleep arousal and/or obstructive sleep apnea. The intraoral appliance can also include an electronics circuit, e.g., having a pulse generator powered by a power source such as a rechargeable battery. The stimulation in some representative embodiments, can be timed with respect to the patient's respiration to maintain upper airway patency or tone during sleep in order to reduce the occurrence of apnea events and/or sleep arousal related to upper airway obstruction and/or OSA. The appliance can also include sensors that are used to trigger the stimulation and/or to determine a response to the stimulation and/or other patient conditions. A “patient” as used herein can refer to a person using the device that may be, but is not necessarily, under the care of a physician.

A representative intraoral appliance can include flexible resilient extensions coupled to more rigid attachment structures or anchors of the intraoral appliance. The flexible resilient extensions can moveably position electrodes adjacent to target nerve branches and/or other target stimulation tissue to maintain effective contact with target structures while in use.

2. Representative Stimulation Targets

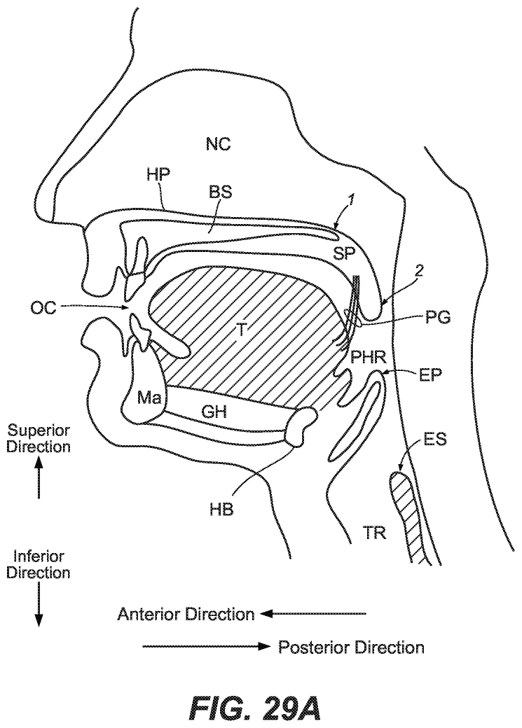

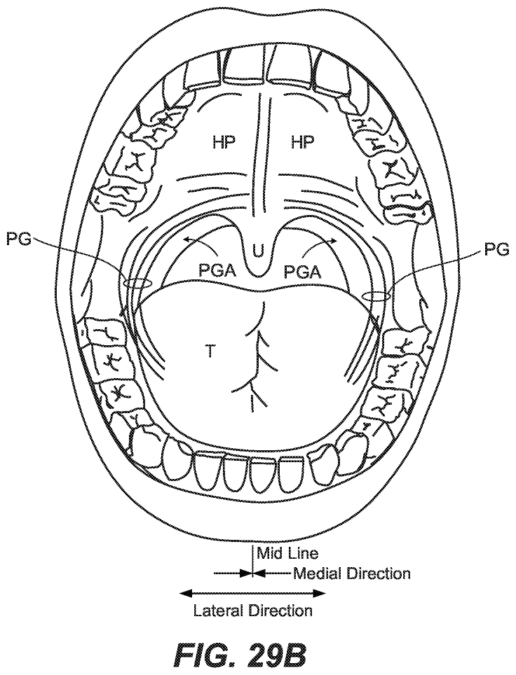

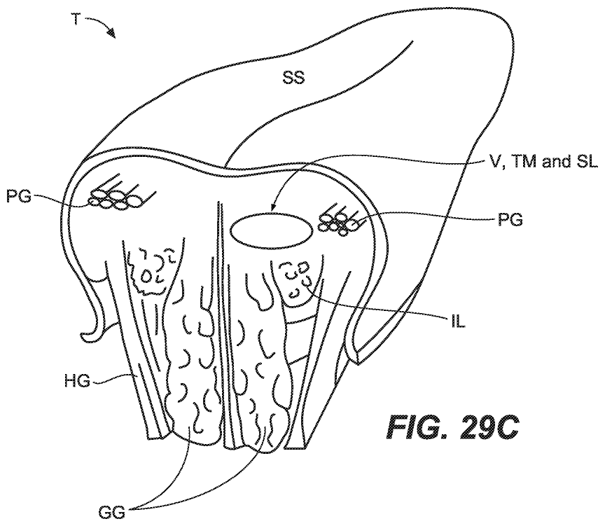

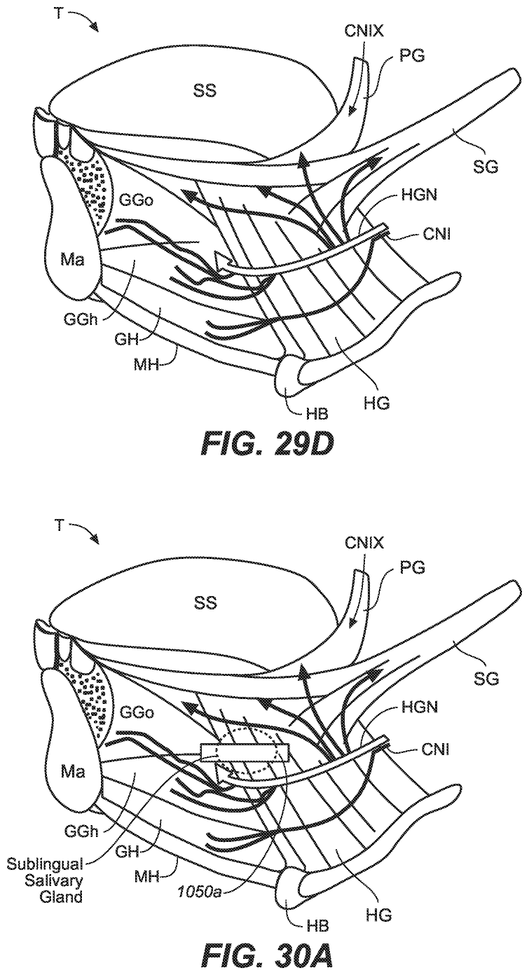

Representative embodiments described herein include an intraoral device that can position electrodes in a target location and/or position, e.g., adjacent a nerve and/or muscle tissue within the oral cavity, for example, as described with reference to FIGS. 29A-29D. The target location and/or position of the electrodes can be identified with respect to a patient's anatomy to direct current through tissue in a manner that provides a desired response to the stimulation. Different electrode positions or locations can be used to target different areas, anatomical structures, and/or tissue. For example, representative target locations can include a location on the tongue and/or adjacent or near nerve endings of the hypoglossal nerve and/or other nerves, with the stimulation effect of moving the tongue and/or other soft tissue to improve upper airway patency and/or improved muscle tone or stiffening. According to some representative embodiments, the target location may be within the sublingual sulcus and directed towards nerve roots that may activate the genioglossus muscle and/or the geniohyoid muscle. The target location can be with respect to any of, or any combination of intrinsic or extrinsic muscles and/or associated nerve branches. Such a target location and/or position can also be distal from the salivary glands (e.g., medial to the sublingual salivary gland) and/or other structures to avoid causing pain and/or other undesired effects. According to some aspects of the present technology, such a target location and/or position can include a target angle or orientation so as to direct current to targeted nerve fibers. Location of the electrode as used herein includes an area, region or position with respect to a patient's intraoral anatomical structures, so as to direct current to tissue in a manner that causes a desired stimulation response.

While a patient is sleeping, the soft tissue of the upper airway and the tongue may move in a manner that creates an upper airway obstruction and/or reduces upper airway patency. The tongue may move, for example, while swallowing. In response to electrical stimulation, the patient's soft tissue and/or tongue also move. Accordingly, the flexible extensions of the intraoral appliance can moveably position the electrodes in a manner that accommodates such movement, while maintaining electrical contact with the target tissue to deliver therapeutic electrical stimulation.

To more fully understand the disclosed embodiments, FIGS. 29A-29D illustrate anatomical elements of a patient's upper airway (e.g., including the nasal cavity, oral cavity, and pharynx of the patient). Accordingly, FIGS. 29A-29D illustrate a number of suitable stimulation targets.

Referring first to FIGS. 29A-29B, the hard palate HP overlies the tongue T and forms the roof of the oral cavity OC (e.g., the mouth). The hard palate HP includes bone support BS, and thus does not typically deform during breathing. The soft palate SP, which is made of soft material such as membranes, fibrous material, fatty tissue, and muscle tissue, extends rearward (e.g., in a posterior direction) from the hard palate HP toward the back of the pharynx PHR. More specifically, an anterior end 1 of the soft palate SP is anchored to a posterior end of the hard palate HP, and a posterior end 2 of the soft palate SP is unattached. Because the soft palate SP does not contain bone or hard cartilage, the soft palate SP is flexible and may collapse onto the back of the pharynx PHR and/or flap back and forth (e.g., especially during sleep).

The pharynx PHR, which passes air from the oral cavity OC and the nasal cavity NC into the trachea TR, is the part of the throat situated inferior to (below) the nasal cavity NC, posterior to (behind) the oral cavity OC, and superior to (above) the esophagus ES. The pharynx PHR is separated from the oral cavity OC by the palatoglossal arch PGA, which runs downward on either side to the base of the tongue T. Although not shown for simplicity, the pharynx PHR includes the nasopharynx, the oropharynx, and the laryngopharynx. The nasopharynx lies between an upper surface of the soft palate SP and the wall of the throat (i.e., superior to the oral cavity OC). The oropharynx lies behind the oral cavity OC, and extends from the uvula U to the level of the hyoid bone HB. The oropharynx opens anteriorly into the oral cavity OC. The lateral wall of the oropharynx includes the palatine tonsil, and lies between the palatoglossal arch PGA and the palatopharyngeal arch. The anterior wall of the oropharynx includes the base of the tongue T and the epiglottic vallecula. The superior wall of the oropharynx includes the inferior surface of the soft palate SP and the uvula U. Because both food and air pass through the pharynx PHR, a flap of connective tissue called the epiglottis EP closes over the glottis (not shown for simplicity) when food is swallowed to prevent aspiration. The laryngopharynx is the part of the throat that connects to the esophagus ES, and lies inferior to the epiglottis EP.

Referring also to FIGS. 29C-29D, the tongue T includes a plurality of muscles that may be classified as either intrinsic muscles or extrinsic muscles. The intrinsic muscles, which lie entirely within the tongue T and are responsible for altering the shape of the tongue T (e.g., for talking and swallowing), include the superior longitudinal muscle SL, the inferior longitudinal muscle IL, the vertical muscle V, and the transverse muscle TM. The superior longitudinal muscle SL runs along the superior surface SS of the tongue T under the mucous membrane, and may be used to elevate, retract, and deviate the tip of the tongue T. The inferior longitudinal muscle IL lines the sides of the tongue T, and is attached to the styloglossus muscle SG. The vertical muscle V is located along the midline of the tongue T, and connects the superior and inferior longitudinal muscles together. The transverse muscle TM divides the tongue at the middle, and is attached to the mucous membranes that run along the sides of the tongue T. The intrinsic muscles are innervated by branches of the hypoglossal nerve.

The extrinsic muscles that attach the tongue T to other structures and are responsible for repositioning (e.g., moving) the tongue, include the genioglossus muscle GG, the hyoglossus muscle HG, the styloglossus muscle SG (FIG. 29D), and the palatoglossus muscle PG. The genioglossus muscle GG is made up of several muscle fibers including the horizontal fibers of the genioglossus horizontal GGh and the oblique fibers of the genioglossus oblique GGo. The genioglossus muscle GG may be used to protrude the tongue T and to depress the center of the tongue T. The genioglossus horizontal GGh connects to the mandible in the anterior, and the back of the tongue in the posterior, where it interdigitates with other muscles of the tongue. When activated, the genioglossus horizontal pulls the bulk of the tongue, including the tongue base, forward. The activation of the genioglossus horizontal GGh may also have the effect of pulling the soft palate forward. The hyoglossus muscle HG may be used to depress the tongue T. The styloglossus muscle SG may be used to elevate and retract the tongue T. The palatoglossus muscle PG may be used to depress the soft palate SP and/or to elevate the back (posterior portion) of the tongue T. The extrinsic muscles of the tongue T described above, except for the palatoglossus muscle PG, are innervated by branches of hypoglossal nerve HGN. The palatoglossus muscle PG is innervated by the pharyngeal branch of the vagus nerve (or CNIX).

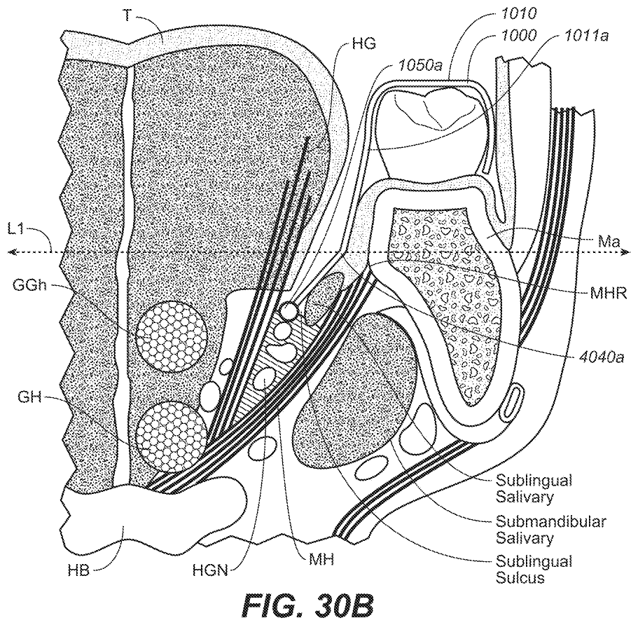

The geniohyoid muscle GH, is also shown in FIG. 29D above the mylohyoid muscle MH. The geniohyoid muscle GH also connects from the mandible Ma to the hyoid bone HB. When the geniohyoid GH contracts, it pulls the hyoid bone HB forward and opens the lower portion of the airway including the epiglottis EP which is one of the collapsed areas in an OSA patient. The geniohyoid muscle GH is stimulated by the CNI branch of the nerve that follows the hypoglossal nerve (and can be considered to be part of the hypoglossal nerve). The mylohyoid muscle MH connects from the hyoid bone HB to the mylohyoid ridge MHR (FIG. 30B) of the mandible Ma.

During awake periods, the muscles of the upper airway (as well as the hypoglossal nerve) are inherently active and stimulated, and may maintain upper airway patency or tone by preventing the soft palate SP from collapsing and/or by preventing the tongue T from prolapsing onto the back of the pharynx PHR. However, during sleep periods, a relatively relaxed state of the soft palate SP may allow the soft palate SP to collapse and obstruct normal breathing, and a relatively relaxed state of the tongue T may allow the tongue T to move in a posterior direction (e.g., onto the back of the pharynx PHR) and obstruct normal breathing.

The directions and/or positions referred to herein with respect to the structures of the intraoral appliance typically refer to anatomical directions or locations when the intraoral appliance is positioned in a patient's oral cavity. The medial-lateral direction is generally the x direction as shown in various Figures herein. The posterior-anterior direction is generally the y direction as shown in various Figures herein. The superior-inferior direction is generally the z direction as shown in various Figures herein. Accordingly, the sagittal plane is the y-z plane, the coronal plane is the x-z plane and the axial plane is the x-y plane.

3. Representative Appliances

Flexible resilient extensions described herein can be used to position electrodes adjacent to and/or in electrical contact with specific locations or anatomical structures in a patient's oral cavity while permitting controlled flexibility and movement. A flexible resilient extension as used herein can, among other things, include or operate as a tether, a position stabilizer, a strut, a support and/or an electrode positioning element. The flexible resilient structure can have one or more arms extending from the attachment structure (e.g., to operate as an anchor). The flexible resilient extension can allow desired movement during use while limiting such movement to maintain the target electrode positioning. The flexible resilient extension can include one or more resilient or spring elements that permit movement of the flexible extension in anterior/posterior directions, while biasing electrodes toward the target tissue for stimulation. The flexible/resilient characteristics of the extensions can also permit the extensions to move in superior/inferior directions and/or medial/lateral directions, while biasing electrodes toward the target tissue.

The one or more flexible resilient extensions can provide controlled flexibility with respect to the angular orientation of the electrodes. The one or more flexible resilient extensions can permit electrodes to roll or rotate about the y axis while controlling or restricting such electrode movement. The one or more flexible extensions can permit the electrodes to pitch or rotate about the x axis while also controlling or restricting such electrode movement. The one or more flexible extensions can permit the electrodes to yaw or rotate about the z axis while also controlling or restricting such electrode movement.

In addition to allowing the electrode(s) to move with the patient's movements, the controlled flexibility of the extensions also allows for proper seating of the electrodes in or at a target location. For example, a representative target location includes the sublingual sulcus, which is an anatomical fold or pocket in which the electrodes may be seated. Some controlled flexibility (including, but not limited to, that with respect to the angular orientation of the electrodes) can permit or enhance positioning the electrode(s) within the sublingual sulcus. Also, the controlled flexibility of the extensions can direct moving elements of the appliance to soft tissue regions where movement occurs and away from harder tissue where moving elements can cause patient discomfort.

The flexible resilient extensions can include struts that limit linear movement in one or more directions and may also limit angular movement such as roll, pitch, yaw or any combination thereof. The flexible resilient extensions can include a resilient material and/or resilient (e.g., spring) elements. The resilient materials in some representative embodiments can be biased or can bias or control movement of the flexible resilient extensions in an inferior direction and/or in a medial direction with respect to the lateral segment to which the extension is coupled. The flexible resilient extensions, which can include resilient elements, in some representative embodiments can be biased or can bias or control movement of the flexible resilient extensions in an anterior direction or a posterior direction, depending on the electrode positioning with respect to desired target anatomical structures, and in order to direct the electrical stimulation current toward target stimulation tissue, areas or regions. In some representative embodiments, the resilient elements can bias the extensions toward an angular orientation. In some representative embodiments, the resilient elements can bias the extensions with respect to a predetermined plane, for example with respect to a sagittal plane, coronal plane, axial plane or a combination thereof. The bias of the structures may also be determined based on the desired position of orientation of the electrodes with respect to anatomical structures.

The flexible resilient extensions described herein may provide flexion points, segments, portions, axes, regions, locations, and/or areas that permit the electrodes to move (within limits) in a variety of directions, e.g., medial-lateral, anterior-posterior, superior-inferior directions, angular orientations, and/or combinations thereof. The flexible resilient extensions can include segments, portions, locations, regions, areas and/or flexion points and/or axes that have a relatively higher flexibility than that of the anchor structure (or other more rigid portions) of the attachment body. In combination, the more rigid anchor structure can be used to prevent electrode movement in particular regions while the flexible resilient extensions can allow movement in particular regions. The flexible resilient extensions may comprise some segments, portions, locations, regions, areas or flexion points or axes that have a relatively higher flexibility than that of other more rigid portions of the flexible resilient extensions. Portions of the flexible resilient extensions can be reinforced with more rigid structures and/or can have a greater material thickness to provide a stiffer or more rigid region of the flexible resilient extension. Flexible resilient extensions herein can optionally include cut-outs, notches, openings or split struts, that permit additional desired flexion of the extensions (or flexion points) and movement of the extensions or electrodes. The cut-outs, notches, openings or split structures can also allow the extensions to avoid certain anatomical structures (for example, salivary glands).

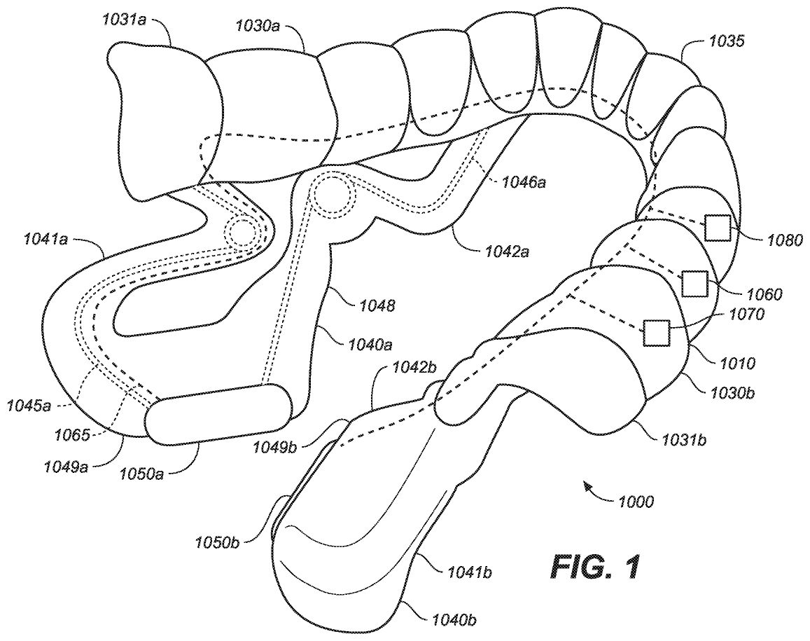

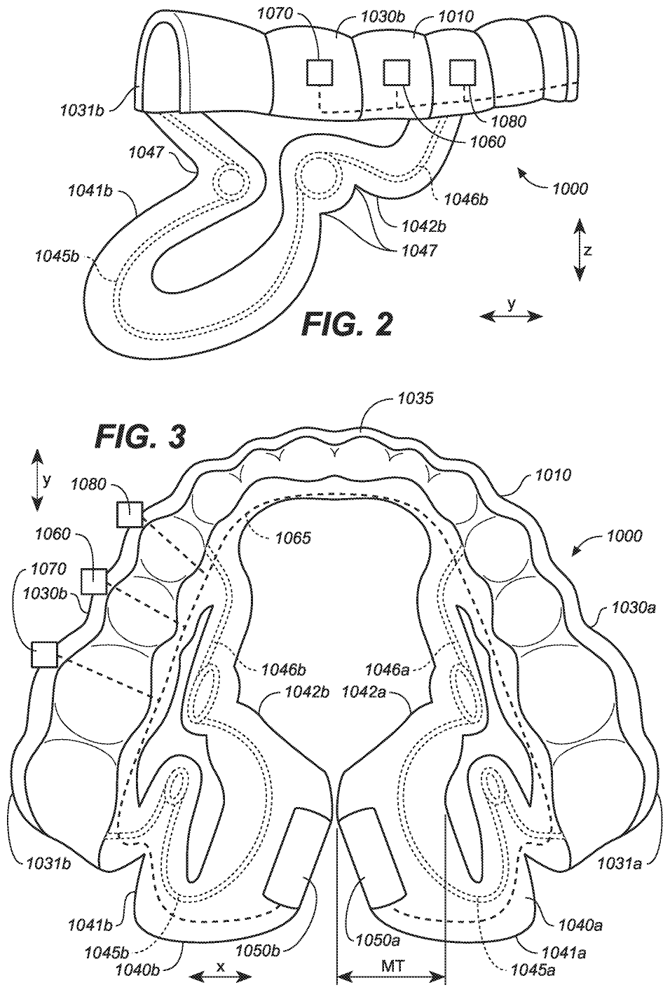

FIGS. 1-3 illustrate a representative removeable intraoral appliance 1000. The intraoral appliance 1000 can include an attachment body 1010 configured to secure the intraoral appliance 1000 within an oral cavity of a patient. The attachment body 1010 can include one or more lateral portions or lateral segments 1030a, 1030b. The lateral segments 1030a, 1030b, in some representative embodiments, can be coupled to each other at a medial location of an anterior portion 1035 of the attachment body 1010. Each lateral segment 1030a, 1030b, respectively, can include a posterior, inferior molar portion 1031a, 1031b that is configured to secure the intraoral appliance 1000 to, or adjacent to, the posterior inferior molars of the patient. For example, a lateral segment can include an attachment element (e.g., an attachment structure or anchor) that is configured to attach or affix the intraoral appliance to one or more of the patient's teeth. The attachment element of the attachment body 1010 can be or can include a structure for example, that is molded or formed, in whole or in part, to fit over the patient's teeth to secure the appliance to one or more teeth. The attachment element can be positioned on only one side of the cavity or can be bilateral. The attachment element can be one of a plurality of attachment elements, e.g., one on each side of the mouth. A brace, clip, or retainer-like structure can be used to anchor the appliance to the teeth, and/or the friction between the attachment element and the teeth can provide this function. The attachment element can be rigid or can have some flexibility. In some representative embodiments, the attachment element can be more rigid than the extension member that positions an electrode in the oral cavity, so as to anchor at least part of the attachment element relatively immovably within the oral cavity. The attachment element may or may not be customized for an individual patient, depending on the implementation.

Flexible, resilient extensions (also referred to herein as extension members) 1040a and 1040b are respectively coupled to the lateral segments 1030a and 1030b at the respective posterior molar portions 1031a, 1031b of the attachment body 1010. The flexible resilient extensions 1040a, 1040b extend inferior and medial of the lateral segments 1030a, 1030b. The flexible resilient extensions 1040a, 1040b can comprise a soft or relatively lower durometer material (e.g., lower than the lateral segments 1030a, 1030b), forming a tissue interface portion 1048.

The flexible resilient extensions 1040a, 1040b can each include a posterior arm 1041a, 1041b and an anterior arm 1042a, 1042b. The posterior arms 1041a, 1041b are coupled to the corresponding posterior molar portions 1031a, 1031b of the lateral segments 1030a, 1030b. The anterior arms 1042a, 1042b are coupled to the attachment body 1010 at a position anterior to the posterior arms 1041a, 1041b. The flexible resilient extensions 1040a, 1040b can include a plurality of bends, curves notches or other flexion points 1047 that permit flexion and/or operate to relieve stress on the extension when experiencing movement within the oral cavity. The arms can also include or can operate as struts to provide structural support and/or as tethers to restrict movement.

Electrodes 1050a, 1050b are coupled to the corresponding extensions 1040a, 1040b at inferior-medial ends 1049a, 1049b of the extensions where the posterior arms 1041a, 1041b join the anterior arms 1042a, 1042b. The posterior arms 1041a, 1041b couple the respective electrodes 1050a, 1050b to the respective posterior molar portions 1031a, 1031b of the corresponding lateral segments 1030a, 1030b. The anterior arms 1042a, 1042b also couple the electrodes 1050a, 1050b to the lateral portions 1030a, 1030b.

The medial ends 1049a, 1049b are sized to have a medial thickness (MT, shown in FIG. 3) that fills a medial-lateral space under a patient's tongue to limit and/or reduce medial-lateral movement of the electrodes 1050a, 1050b when positioned in the oral cavity.

As shown in FIGS. 1-3, first resilient elements (e.g., springs) 1045a, 1045b can be incorporated into the posterior arms 1041a, 1041b. They can be attached, encased or otherwise integrated with the flexible resilient extensions 1040a, 1040b. Second resilient elements 1046a, 1046b are respectively incorporated into the anterior arms 1042a, 1042b. Any of the foregoing resilient elements can bias the extensions 1040a, 1040b in a variety of directions in order to direct the electrodes toward contact with the target stimulation tissue, areas or regions. For example, the extensions can be biased in a medial, inferior and/or posterior direction. In some representative embodiments, the resilient elements can bias the extensions to or toward an angular orientation, for example, with respect to a predetermined plane, including but not limited to, with respect to a sagittal plane, coronal plane or medial plane or a combination thereof. The first resilient elements 1045a, 1045b can bias the posterior arms so that the attached electrode contacts tissue for stimulation. Likewise, the second resilient elements 1046a, 1046b can bias the anterior arms so that the attached electrode contacts tissue at the target location. In some representative embodiments, the second resilient elements 1046a, 1046b can be configured to bias the extensions 1040a, 1040b in a posterior direction so that if the tongue moves forward and pushes the extensions 1040a, 1040b forward, the extensions 1040a, 1040b will tend to move posteriorly toward their original positions.

The extensions 1040a, 1040b can be configured to be more rigid in one direction than another. For example, the flexible extensions 1040a, 1040b can be relatively more rigid in a medial-lateral direction than in the anterior-posterior direction. Such a configuration can provide more consistent tissue contact and direction of current flow by allowing more movement in the anterior-posterior directions when the tongue moves forward and back while maintaining contact against tissue at a target location. In some representative embodiments, the flexible extensions 1040a, 1040b can also be relatively more rigid in a medial-lateral direction than in an inferior-superior direction. In some representative embodiments, the flexible extensions 1040a, 1040b can also be relatively more rigid in an inferior-superior direction than in an anterior-posterior direction.

The posterior arms 1041a, 1041b and the anterior arms 1042a, 1042b together can guide, limit and/or control the movement of the electrodes 1050a, 1050b while the electrodes are positioned in the oral cavity of a patient. Accordingly, the extensions 1040a, 1040b, the posterior arms 1041a, 1041b and the anterior arms 1042a, 1042b can position the electrodes 1050a, 1050b at a target location with respect to the patient's anatomy to direct current through tissue to provide a desired stimulation response.

According to representative embodiments, the posterior arms 1041a 1041b can limit movement in an anterior direction while the anterior arms 1042a, 1042b can limit movement in a posterior direction. The arms can also limit angular movement such as roll, pitch, yaw or any combination thereof. Thus, the extensions can allow movement, for example of the tongue and surrounding soft tissue, and the electrodes within the oral cavity, while allowing but limiting and controlling the movement of the extensions 1040a, 1040b and therefore the electrode position.

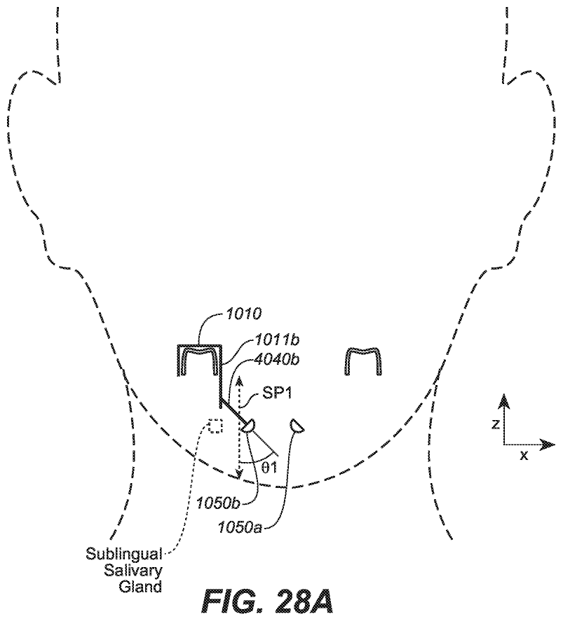

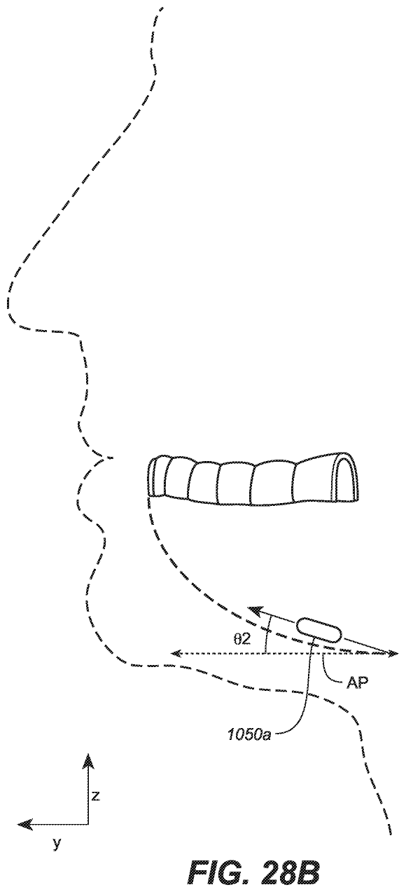

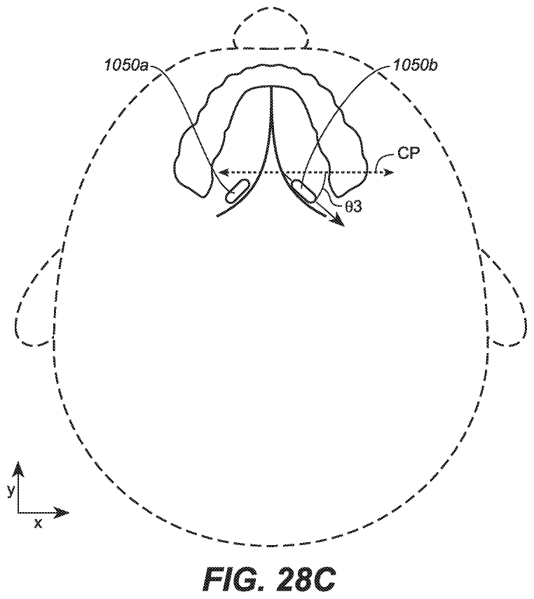

The electrodes 1050a, 1050b are shown oriented at approximately 45 degrees with respect to a sagittal plane (or a y-z plane, as described later with reference to FIGS. 28A-28C). However, the electrodes can be oriented at other angles, as is also described in more detail later. The orientation of the electrode angle shown or described herein, in some representative embodiments is an “original angle”, i.e., the angle that the electrode is at prior to being positioned in the oral cavity. When positioned in the oral cavity, the angle may change due to contact between the appliance 1000 and the anatomical features of a patient's oral cavity. However, in representative embodiments, the electrodes are biased toward the original angular orientation.

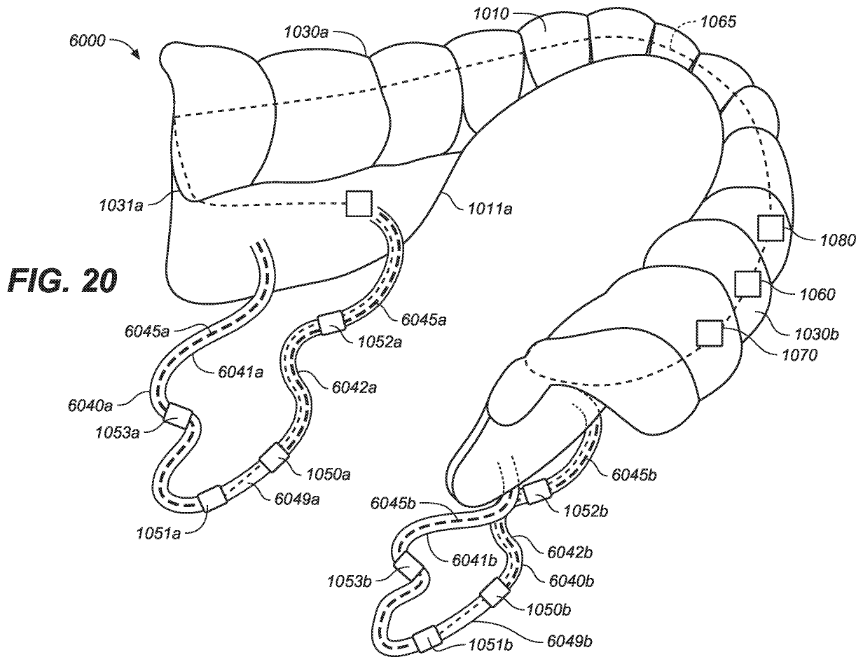

The electrodes 1050a, 1050b can have a variety of suitable shapes and/or sizes. The electrodes can include flat or rounded portions or arced surfaces to enhance (e.g., optimize) tissue contact and stimulation response. The electrodes 1050a, 1050b can include a single electrode carried by each lateral segment 1030a, 1030b, or a plurality of electrodes (e.g., an array) that may be selected (individually or as set or subset) for a target stimulation response. FIGS. 20, 21, and 27, described in further detail later, illustrate representative arrays.

In some embodiments, the intraoral appliance 1000 is customized to fit a particular patient's oral cavity. For example, the elements forming the appliance 1000 can be specifically sized and/or shaped to provide tissue contact at a particular patient's anatomical location, and/or in a location that is identified to provide a desired therapeutic response in a particular patient. The intraoral appliance can be further customized to provide more efficient and/or better-directed electrical stimulation to the oral cavity tissue of an individual patient. The customized attachment body can be constructed from a mold, or can be 3D printed to conform or fit on one or more inferior teeth of the particular patient, accounting for the particular patient's bite.

The attachment bodies described herein with respect to the various Figures can be constructed from a variety of suitable materials, including ethylene vinyl acetate, polycarbonate, nylon, and/or other thermoplastics. The soft or relatively low durometer material forming the tissue interfaces described herein can include, for example, a silicone, urethane, polyurethane, and/or polyurethane foam. The flexible resilient structures described herein can include an elastic material, a resilient material, and/or a spring material such as, for example, stainless steel, nitinol and/or a combination of materials having suitable flexibilities and rigidities. In addition, the flexible resilient structures can have a varying flexibility and/or other mechanical properties, along the length of the structures. The resilient elements can be incorporated into the extensions in a number of manners including but not limited to being embedded in, attached or otherwise coupled to, injected into, or otherwise formed with the extensions. In some embodiments, a low durometer material is combined with a resilient structure, material or spring element or material to provide a soft exterior or tissue interface.

A practitioner or manufacturer can identify a predetermined electrode position to target a particular tissue or tissue region in the patient's oral cavity. In some embodiments, a customized device can then target such tissue, with the customized parameters including but not limited to, electrode position, electrode angle, extension dimensions, strut dimensions, flexion point locations and/or flexion directions. Such customized device parameters can be based on a preliminary test of the patient's response to various stimulation parameters, device geometry parameters, and/or the patient's own particular anatomy.

As shown in FIGS. 1-3, the intraoral appliance can further include an electronics circuit 1060 (shown schematically) optionally including a pulse generator, logic circuitry and/or a controller. The electronics circuit is electrically coupled by way of one or more electrical connections 1065 (e.g., wires) to one or more electrodes 1050a, 1050b (also referred to herein with reference numbers 1051a, 1051b) and configured to deliver electrical stimulation through tissue within the oral cavity of a patient.

The intraoral appliance 1000 can further include a sensor 1070 configured to sense biometric information corresponding to one or more patient parameters including, but not limited to, respiration parameters (e.g., inhalation and exhalation cycles/waveforms), sleep arousal, pulse oxygen, oxygen saturation, heart rate, body temperature, stimulation response parameters, apnea events, body position, jaw, tongue, soft tissue movement or position, and/or other patient movement or position, tongue location, location of tongue with respect to mouthpiece, nose breathing versus mouth breathing, detection of when in a breathing cycle mouth versus nose breathing occurs, detection of rescue breaths, and/or other parameters indicative of conditions of the patient or the patient's upper airway/oral cavity. Sensors, for example, can include but are not limited to, temperature sensors such as thermistors and/or thermocouples, sound sensors, vibration sensors, pressure sensors, force sensors, strain gauges, magnetometers, accelerometers, gyroscopes, impedance sensors, EMG sensors, gas sensors and/or chemical sensors, oxygen saturation sensors, and/or other sensors that can sense conditions of the patient. In some representative embodiments, the patient's respiration parameters can be used to trigger stimulation based on the patient's breathing cycle as well as information that may indicate an apnea event is occurring or is likely to occur.

This information obtained from the sensor(s) can be used to determine when to stimulate. For example, electrical stimulation can be provided to the patient immediately prior to inhalation to ensure upper airway patency or tone during inhalation. Stimulation can be provided at other times as well, for example at the end of exhalation and into an inhalation cycle, or when an apnea event is detected. Stimulation can also be triggered by other parameters. In some representative embodiments, for example, stimulation can be triggered by sensing tongue position or movement. Stimulation can also be generally constant, e.g., always on. When on, the applied electrical current can be applied to different electrodes, e.g., in a repeated cycle.

Sensors can also be used to detect the patient's response to stimulation and can be used to adjust the stimulation parameters, including which electrode(s) are active at any particular time. EMG sensors can also be used to sense muscle contraction or force to determine the patient's response to stimulation. Impedance sensors on the mouthpiece can sense the location of the tongue with respect to the mouthpiece, extension or electrodes. In addition to or in lieu of the foregoing functions, one or more sensors can be used to determine system performance, electrode/tissue contact and/or effectiveness, and/or movement of the electrodes and/or extensions coupled to the electrodes.

The overall system can include logic circuitry to control one or more aspects of the electrical stimulation provided to the patient via the intraoral appliance. The logic circuitry can be programmed to determine or select target stimulation parameters, to assess the patient's response to the stimulation, to select a stimulation protocol (e.g., including which electrodes are active, and when), to use sensed information to initiate, adjust, modify and/or cease stimulation, and/or to transmit, receive and/or record data related to treatment or patient condition or related stimulation parameters. A power source, e.g., a battery 1080 can provide power to the pulse generator, sensors, controller and/or logic circuits and can be replaceable or rechargeable. One or more electrodes described herein can be used to sense information from within the patient's oral cavity such as, for example, via EMG or detecting NCV (nerve conduction velocity), or for detecting impedance. The logic circuit can include a controller programmed with a logic program configured to receive input from the sensor(s) and to control the stimulation delivered to the patient in response to one or more logic conditions.

Various representative embodiments throughout this application may be shown with elements having the same or similar reference numbers as elements that are described in FIGS. 1-3 or other Figures, and accordingly can have the same or generally the same characteristics and operation as described with reference to FIGS. 1-3 or such other Figures.

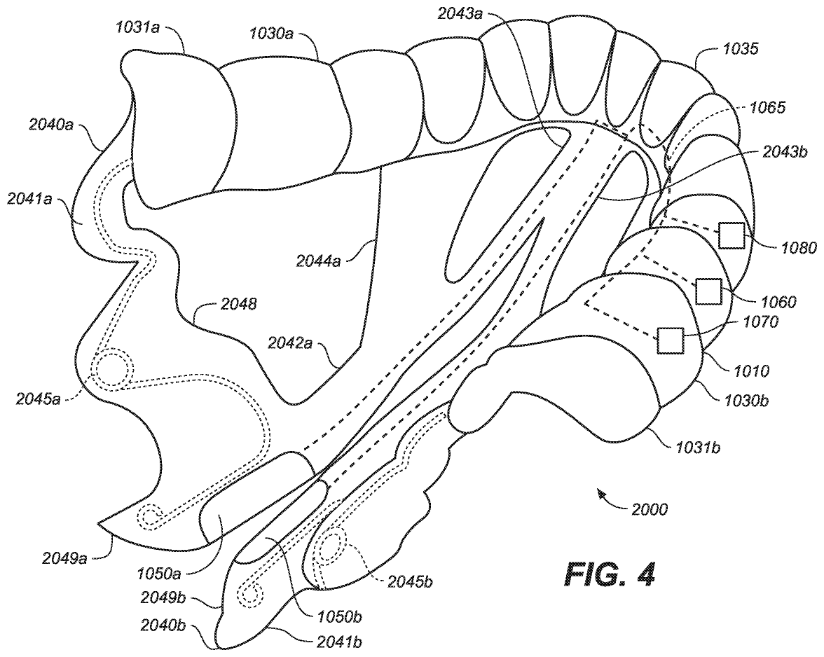

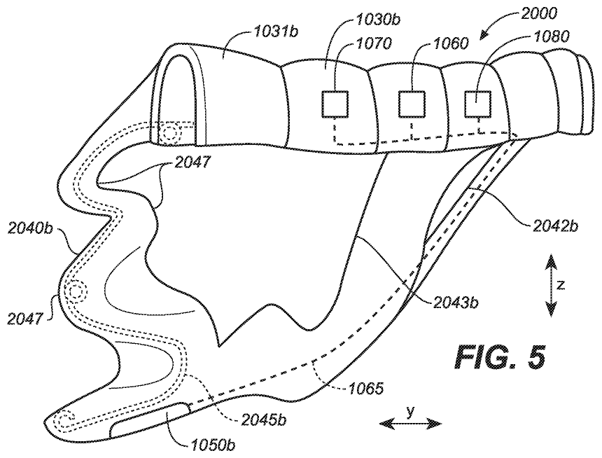

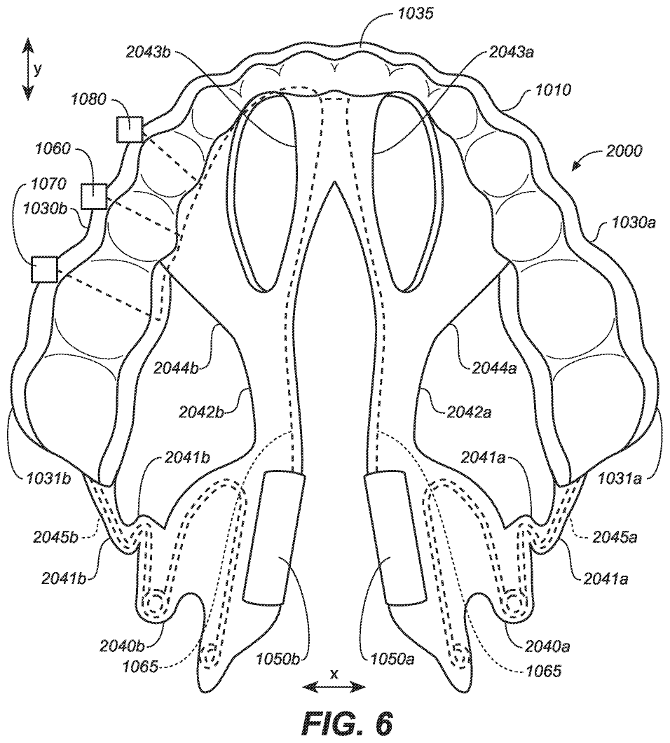

FIGS. 4-6 illustrate a removeable intraoral appliance 2000 configured in accordance with some embodiments of the present technology. The intraoral appliance 2000 includes an attachment body 1010. Flexible, resilient extensions 2040a and 2040b are respectively coupled to the corresponding lateral segments 1030a and 1030b at the corresponding posterior molar portions 1031a, 1031b of the attachment body 1010. The flexible resilient extensions 2040a, 2040b can extend inferior and medial of the lateral segments 1030a, 1030b. The flexible resilient extensions 2040a, 2040b can include a patient interface portion 2048 that includes a relatively lower durometer material. The flexible resilient extensions 2040a, 2040b comprise a plurality of bends, curves, notches or other flexion points 2047.

The flexible resilient extensions 2040a, 2040b can each include a posterior arm 2041a, 2041b and an anterior arm 2042a, 2042b. The posterior arms 2041a, 2041b are coupled to posterior molar portions 1031a, 1031b of the lateral segments 1030a, 1030b. The anterior arms 2042a, 2042b are coupled to the attachment body 1010 at a location anterior to the posterior arms 2041a, 2041b. The anterior arms 2042a and 2042b can include anterior branches 2043a, 2043b and lateral branches 2044a, 2044b, respectively. The anterior branches 2043a, 2043b are attached to the anterior segment or location 1035 in a location anterior to the posterior molar portions 1031a, 1031b. The anterior branches 2043a, 2043b can also be attached to each other. The lateral branches 2044a, 2044b connect the anterior arms 2042a, 2042b to the lateral segments 1030a, 1030b.

Electrodes 1050a, 1050b are coupled to the corresponding extensions 2040a, 2040b at the inferior-medial ends 2049a, 2049b where the posterior arms 2041a, 2041b and anterior arms 2042a, 2042b are coupled together. As shown in FIGS. 4-6, the resilient elements 2045a, 2045b are incorporated into the posterior arms 2041a, 2041b. The resilient elements can bias the extensions 2040a, 2040b in one or more directions (e.g., medial/lateral, inferior/superior, anterior/posterior directions and/or toward an angular orientation) in order to direct the electrodes toward the target stimulation tissue, areas and/or regions. Extensions 2040a, 2040b may be configured to be more rigid in one direction versus another or to have more permitted motion in one direction versus another.

As further illustrated, the anterior arms 2042a, 2042b do not include resilient elements similar to the resilient elements 2045a, 2045b carried by the posterior arms 2041a, 2041b. The anterior arms 2042a, 2042b are less bulky than the anterior arms of FIGS. 1-3 because they do not include an encased spring member and are accordingly more comfortable to the patient. While the anterior arms 2042a, 2042b do not include spring elements that bias the anterior arms 2042a, 2042b in a posterior direction, they operate as tethers or stabilizers that prevent twisting or other undesired movement (rotational, medial-lateral, inferior-superior and/or superior inferior movement) of the posterior arms 2040a, 2040b and the attached electrodes 1050a, 1050b. The anterior arms 2042a, 2042b in representative embodiments can instead or in addition, include additional spring elements.

The anterior arms 2042a, 2042b each have multiple attachment points (i.e., with anterior branches 2043a, 2043b and lateral branches 2044a, 2044b) to the body 1010 with different locations and orientations that, in combination, resist movement in multiple directions. Connecting the anterior branches 2043a, 2043b of the extension members to the anterior location 1035 of the body can keep the electrodes closer to the frenulum. Connecting the anterior arms 2042a, 2042b together can maintain similar posterior-anterior positions of the electrodes 1050a, 1050b with respect to each other. While increasing control of electrode position and movement, connecting the branches 2042a, 2042b at the anterior location 1035 may prevent posterior movement of the electrodes, such movement which may be desirable to maintain electrode contact when the tongue moves. Additionally, while increasing control of electrode movement, the multiple branches of the anterior arms can increase the number of device contact points under a subject's tongue which may reduce comfort. Such multiple attachment point elements can be further useful in customized devices where anatomical structure of an individual patient may be identified and avoided for patient comfort using a more customized device construction.

In some representative embodiments, the anterior arms 2042a, 2042b can be constructed of a material with resilient properties that allow the arms to more elastically restrain electrode movement. Although not shown, in some representative embodiments resilient elements (e.g., similar to elements 2045a, 2045b) may be included in the anterior arms 2042a, 2042b to provide additional spring bias or structural rigidity in a manner similar to FIGS. 1-3, e.g., if comfortable for the patient and/or required for suitable electrode placement.

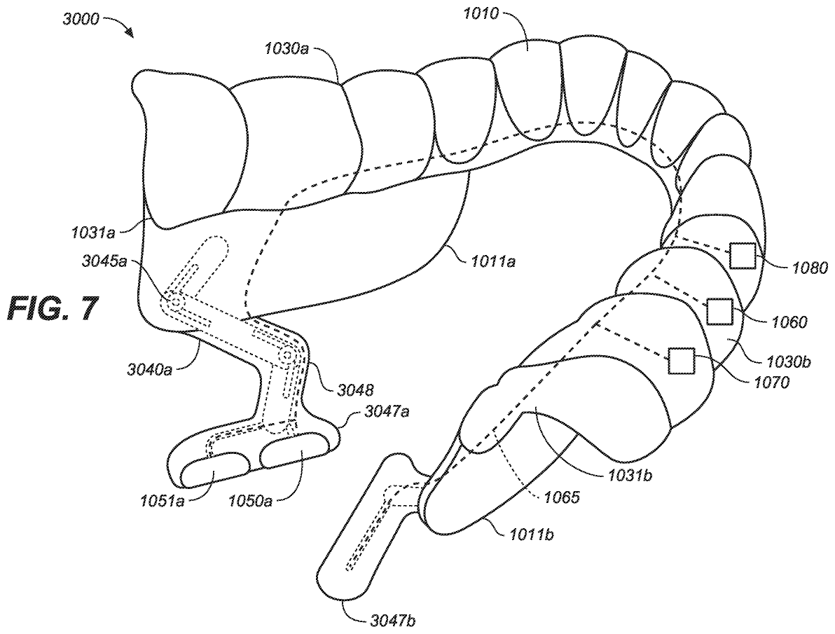

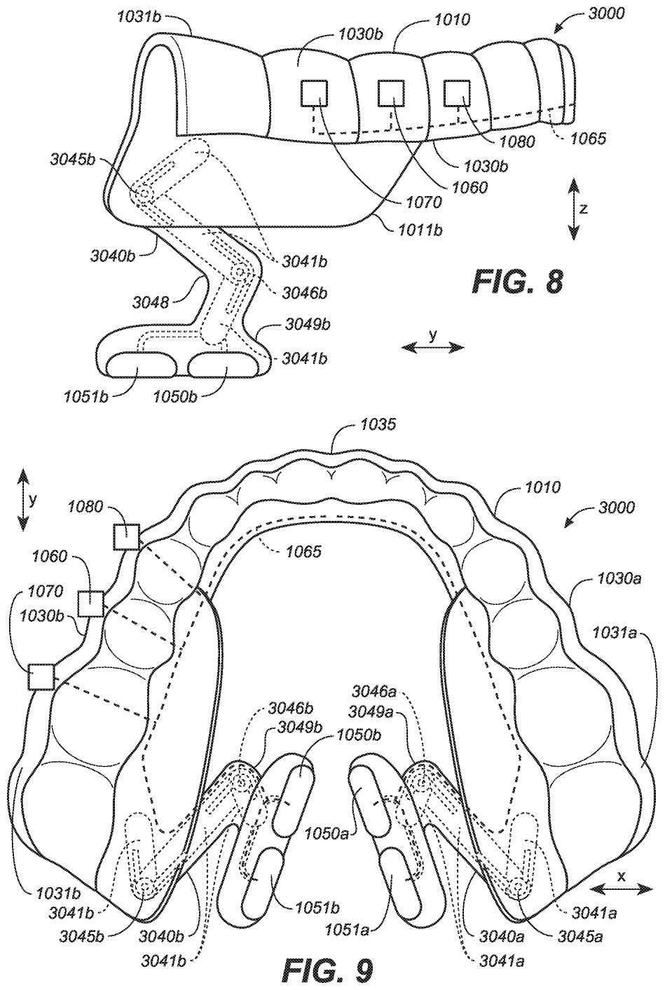

FIGS. 7-9 illustrate another representative removeable intraoral appliance 3000 that includes extensions 3040a, 3040b having an arrangement of flexibly linked rigid segments. In addition, the mouthpiece body 1010 includes inferiorly extending, posterior rigid flaps (or wings) 1011a, 1011b. (See also FIGS. 28A and 30B). The posterior rigid flaps 1011a, 1011b extend in an inferior direction from corresponding posterior molar portions 1031a, 1031b. The flexible, resilient extensions 3040a and 3040b are coupled to corresponding posterior rigid flaps 1011a, 1011b.

In a further aspect of the arrangement shown in FIGS. 7-9, the flexible resilient extensions 3040a, 3040b include a single arm with a plurality of more rigid segments 3041a, 3041b joined by resilient connectors 3045a, 3045b and 3046a, 3046b. The rigid segments 3041a, 3041b and resilient connectors 3045a, 3045b, 3046a, 3046b may be attached, encased or otherwise integrated with the flexible extension structure. For example, the rigid segments 3041a, 3041b and resilient connectors 3045a, 3045b, 3046a, 3046b may be covered with a patient interface portion 3048 that comprises a relatively lower durometer material. The flexible resilient extensions 3040a, 3040b provide a single attachment location to the body 1010.

In a representative arrangement, each extension 3040a, 3040b includes a corresponding plurality of electrodes 1050a, 1051a and 1050b, 1051b coupled to its distal (inferior) end. For example, first electrodes 1050a, 1051a are positioned on one electrode support 3047a which is coupled to the inferior end 3049a of a first extension 3040a, and second electrodes 1050b, 1051b are positioned on another electrode support 3047b which is coupled to the inferior end 3049b of extension 3040b.

The resilient connectors (e.g., springs or other suitable structures) can bias the extensions 3040a, 3040b in a medial/lateral, inferior/superior, and/or anterior/posterior direction, and/or toward an angular orientation, so as to direct the electrodes towards target stimulation tissue, areas or regions. Each connector may move in a different direction and have a different flexibility. The rigid elements 3041a, 3041b can be more rigid than the resilient connectors 3045a, 3045b, 3046a, 3046b. The rigid elements 3041a, 3041b can provide segments of support to the extension members while the resilient connectors 3045a, 3045b, 3046a, 3046b provide flexion points between segments, regions, portions, areas, or locations of the device. The multiple connectors can allow specifically directed or biased movement while the rigid segments can limit the amount of flexion or directions of flexion of segments of the extensions 3040a, 3040b. Accordingly, aspects of both the single arm extension and the combination of rigid and flexible elements can create more simple, predictable electrode movement within the oral cavity. The rigid elements can also prevent undesired movement. The flexible connectors can include spring elements that bias the electrodes toward a desired tissue contact location. The combination of the rigid elements with controlled flexion points may also avoid buckling of the extension members. In addition, the single arm can be more comfortable to a patient (particularly when the patient's mouth/tongue move) because it has fewer elements positioned in the oral cavity and near the tongue.

Conversely, manufacturing the device with rigid elements and flexible connectors may be more complicated due to a greater number of parts and smaller parts, and may require greater manufacturing precision to ensure precise movement. In addition, encasing such parts may make the device bulkier. Also, while the movement is repeatable, the more controlled flexion points may not be suitable for a wide range or variety of oral cavity anatomies that may vary from patient to patient.

The flexible resilient extensions 3040a, 3040b can be attached at, and extend inferior and medial of, the posterior rigid flaps 1011a, 1011b. The posterior rigid flaps 1011a, 1011b can extend from the lateral segments 1030a, 1030b in an inferior direction approximately to or adjacent the mylohyoid ridge MHR (FIG. 30B) of the mandible Ma (FIG. 30B) (a location where the mylohyoid muscle MH attaches to the mandible Ma, and, below which the target soft tissue structures are located). (See also FIGS. 28A and 30B for representative positioning of flaps 1011a, 1011b). To improve comfort, the motion of the flexible components of the extension members can be limited to the areas of soft tissue, avoiding movement in regions of hard tissue. Further, the motion of the flexible connectors can be limited to the locations where strain relief from tissue movement (e.g., tongue movement) is desired.

The range of motion of the extensions can allow the electrodes follow the range of motion of the soft tissue so that it can maintain contact during soft tissue movement, while restricting electrode movement beyond and out of soft tissue contact. Accordingly, the flexible resilient extensions 3040a, 3040b are attached to the posterior rigid flaps 1011a, 1011b so that they can flex in a superior direction within a broad, full, and/or predetermined range of soft tissue below the mandibular ridge, but only so that the electrodes stay within a region of the soft tissue of the oral cavity inferior to the mylohyoid ridge. The posterior rigid flaps 1011a, 1011b can be generally positioned in a region in the oral cavity adjacent the harder tissue of the mandible. The location of the mylohyoid ridge may vary from patient to patient and accordingly the posterior rigid flaps and or the extension flexibility range can be customized for individual patients.

Multiple electrodes on each extension in this and other representative embodiments herein can provide benefits and/or options for the patient and/or practitioner. For example, electrode pairs may be selected from the multiple electrodes for a variety of reasons prior to or during treatment stimulation. Use of different electrodes can provide alternative tissue contact points as well as alternative current paths through the tissue. Different electrodes and/or stimulation parameters can be used to target different areas, anatomical structures, and/or tissue. One or both of electrodes 1050a, 1051a on one side of the appliance may be selected as having a first polarity while one or more of electrodes 1050b, 1051b on the opposite side may be selected as having an opposing polarity where current is directed from one side of the oral cavity to the other through the target tissue. First electrodes 1050a, 1051a, may be used as an electrode pair of opposite polarity located on one side of the oral cavity. Likewise, second electrodes 1050b, 1051b may be used as an electrode pair of opposite polarity on the opposite side of the oral cavity. The pairs may be activated alternately or simultaneously. Electrodes may be selected from electrode arrays, for example, as shown in representative embodiments herein. Electrode selection can, for example, be based on testing prior to use of the appliance or as an adjustment after initial use of an appliance. Electrode selection can also be made during use of the device. Electrode selection can be based on an algorithm, based on sleep lab and/or other response observations, based on sensed stimulation responses, and/or based on movement within the oral cavity (for example, tongue movement that occurs with electrical stimulation and/or a change in body position) and/or other sensed information.

Different waveforms or variations of pulse width, amplitude and frequency may also be selected and/or implemented in a similar manner to that described with respect to electrode selection. For example, as the patient's tongue base widens in a posterior direction, changing the pulse width and amplitude may compensate for the varying thickness of the muscle and/or greater distance between opposing electrodes. Different electrode pairs at different locations can deliver different waveforms. The practitioner can also vary the waveform from patient to patient, and/or use multipolar electrode configurations. Electrodes, stimulation parameters and/or programs may be selected or adjusted prior to and/or after deployment. They may be selected based on response in a testing mode, for example, by observing or sensing responses or during sleep in a sleep lab or similar environment. Patient responses may be observed using visualization or sensing of upper airway parameters, other patient parameters, and/or other appropriate criteria such as the AHI (Apnea, Hypopnea Index). Electrodes and/or stimulation parameters or programs may be selected and/or adjusted during use in response to a position change of the extension members or electrodes. For example, if the tongue moves the electrodes forward, the more posteriorly situated electrodes 1051a, 1051b may be selected. Electrode response may also change during sleep or during treatment due to movement of the electrodes, and/or patient movement and/or position change. Electrode stimulation parameters and/or stimulation programs may be adjusted based on observations in a sleep lab or otherwise during sleep. Further, different electrodes, stimulation parameters and/or stimulation programs may be selected if the patient habituates to a particular form of stimulation, in order to elicit a more effective response.

Electrodes 1050a, 1051a, 1050b, 1051b may be selectively activated via logic or controller circuitry of the circuit 1060. Electrodes may be selected based on feedback from sensors (e.g., sensor 1070) that indicate the efficacy of the patient's response to stimulation, including changes that may occur, for example, due to electrode position change or habituation. Accordingly, the sensors may provide information to the electronic circuitry or logic that will be used to adjust the stimulation parameters, including electrode selection (e.g., which electrode(s) is/are active). According to representative embodiments, a body position sensor may be used to determine the position of a subject and to select electrodes based on body position and expected electrode position based on the sensed body position. For example, if a patient is lying on his or her back, the more posterior electrode may be selected assuming that the tongue may move back in a subject's oral cavity in this position. Further, for example, if a left side patient position is sensed, the left electrode pair may be selected for stimulation. In some embodiments, one or more dedicated sensors 1070 provide feedback to control the therapy delivery process. In some embodiments, in addition to or in lieu of the dedicated sensor(s) 1070, the electrodes 1050a, 1051a, 1050b, 1051b, can operate as sensors, as well as therapy signal delivery devices. For example, the electrodes can sense the patient's response during times when the electrodes are not actively delivering an electrical signal. As discussed herein, such breaks in the signal delivery process can occur between pulses of the therapy signal, and/or as a result of the therapy signal being active during only portions of the patient's breathing cycle.

In addition to facilitating electrode selection, multiple electrodes may be used in a program that cycles automatically through patterns of electrodes to improve stimulation results, for example to avoid habituation. Additionally, switching electrode pairs can occur during a single stimulation cycle. For example, during a single breath the electrode selection may switch from electrodes at one location to electrodes at another location. For example, a more anterior electrode may be selected immediately prior to inhalation. And, in order to maintain effective electrode target contact, a posterior electrode may be selected after initiation of inhalation, assuming that the tongue will move the inferior or distal end of the extension forward.

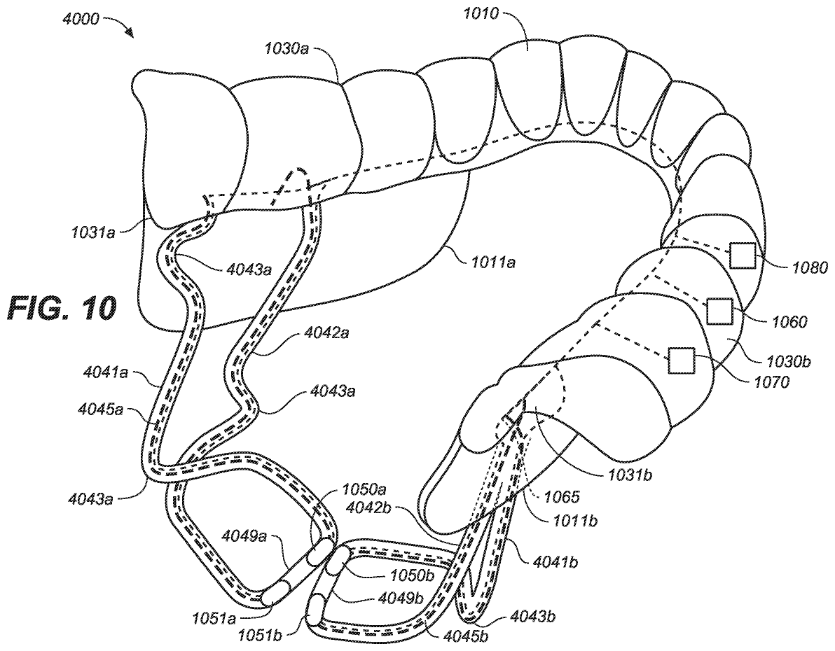

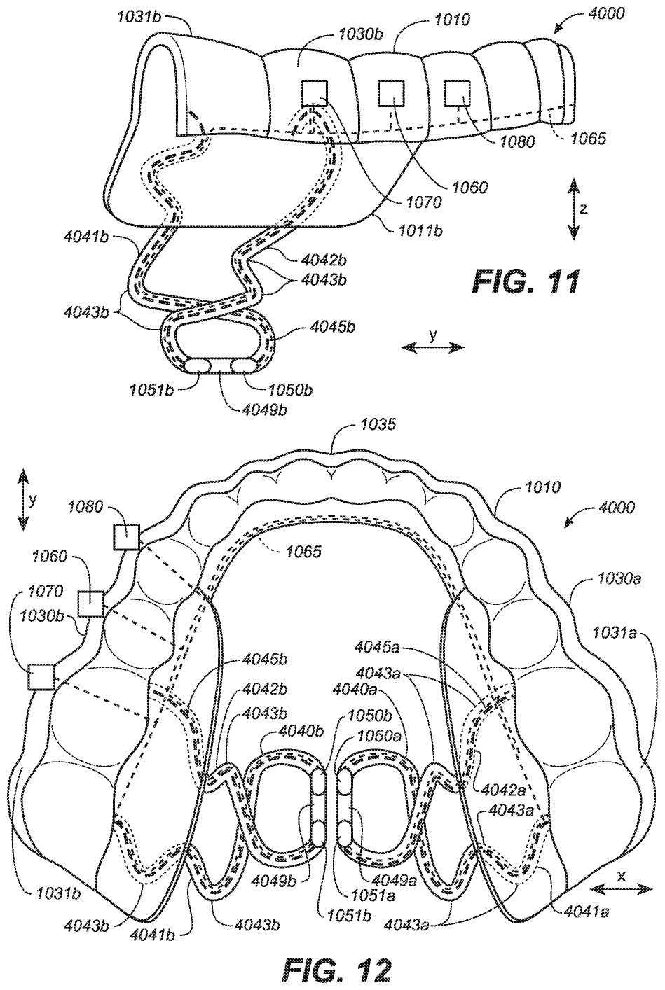

FIGS. 10-12 illustrate a representative removeable intraoral appliance 4000 having an attachment body 1010. Flexible, resilient extensions 4040a and 4040b are coupled to the corresponding flaps 1011a, 1011b which are coupled to lateral segments 1030a and 1030b at the corresponding posterior molar portions 1031a, 1031b. The flexible resilient extensions 4040a, 4040b may extend inferior and medial of the flaps 1011a, 1011b. In some representative embodiments, the flexion points or stress relief points of the extensions 4040a, 4040b can be positioned to limit flexion or movement to locations inferior to the flaps 1011a, 1011b. And, as will be described in further detail below, the extensions can each comprise one or more arms coupled to the attachment body flaps at multiple points with the arms crossing over each other to provide stress relief and flexion points. The resulting shape can also form openings that avoid/limit potentially painful or uncomfortable tissue contact. For example, in FIG. 28A, representative extension member 4040b is shown positioned adjacent a sublingual salivary gland, with the salivary gland seated between arms 4041b, 4042b. Thinner less bulky extension members can provide more comfort to the patient.

First electrodes 1050a, 1051a are positioned on the inferior end 4049a of a first extension member 4040a, and second electrodes 1050b, 1051b are positioned on the inferior end 4049b of a second extension member 4040b. The extension members 4040a, 4040b, respectively, couple the electrodes to the corresponding flaps 1011a, 1011b of the body 1010.

The flexible extensions 4040a, 4040b can each include a corresponding posteriorly originating arm 4041a, 4041b coupled to the flaps 1011a, 1011b, and an anteriorly originating arm 4042a, 4042b coupled to flaps 1011a, 1011b in a location anterior to the posteriorly originating arms 4042a, 4042b. The posteriorly originating arms 4041a, 4041b and anteriorly originating arms 4042a, 4042b can each have a plurality of bends or undulations 4043a, 4043b along their lengths to provide flexion points or regions. Each posteriorly extending arm 4041a, 4041b crosses over a corresponding anteriorly originating arm 4042a, 4042b before attaching or joining at their corresponding inferior ends 4049a, 4049b. The crossed-over arms 4041a, 4042a and 4041b, 4042b can form openings adjacent the inferior ends 4049a, 4049b that can provide relief for anatomical structures. For example, such openings can be positioned adjacent salivary glands to avoid painful contact with the glands. (See FIG. 28A). The crossed over arms 4041a, 4042a and 4041b, 4042b also form stress relief or flexion points.

The arms 4041a, 4041b, 4042a, 4042b can include a flexible material. The flexible material may also be resilient and/or can comprise wires 4045a, 4045b that act as spring elements or stiffening elements that may be encased in a lower durometer material. The arms 4041a, 4041b, 4042a, 4042b are relatively thinner than the arms shown in FIGS. 1-3 and in particular, the inferior ends 4049a, 4049b are thinner and less bulky which allows for greater comfort but permits greater movement of the inferior ends 4049a, 4049b and corresponding electrodes 1050a, 1050b, 1051a, 1051b. According to particular representative embodiments, wires 4045a, 4045b bias the inferior ends 4049a, 4049b in a medial direction toward electrical contact in a target tissue area. Wires 4045a, 4045b can bias any or more of the arms 4041a, 4041b, 4042a, 4042b in a variety of other directions in order to direct the corresponding electrodes toward target stimulation tissue, areas or regions. The wires or other support elements described herein may support the extensions while the cross-over regions, bends or undulations 4043a, 4043b may provide flexion points or regions that allow controlled movement as described with respect to various representative embodiments herein. The arms 4041a, 4041b, 4042a, 4042b may also operate as tethers or range of motion limiters for the extensions and attached electrodes.

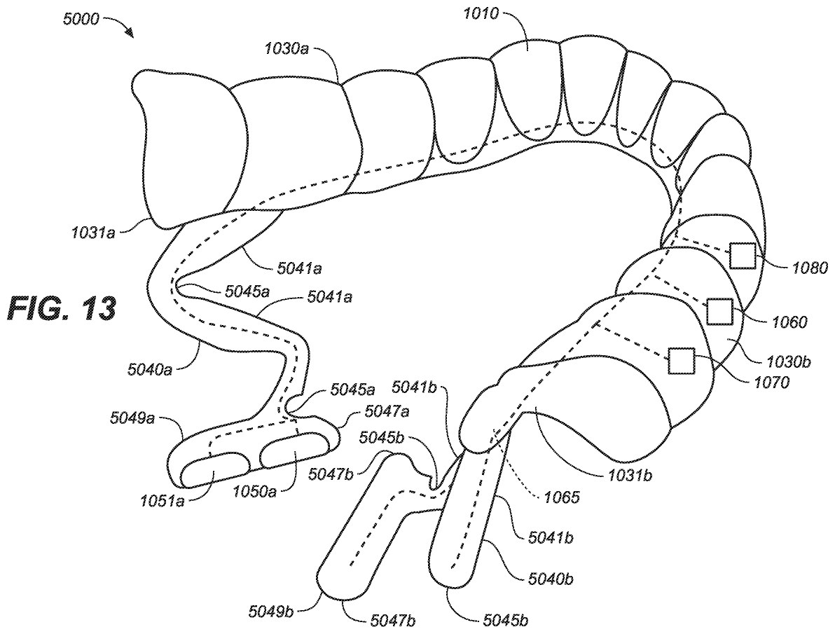

FIGS. 13-15 illustrate a representative removeable intraoral appliance 5000 having an attachment body 1010. The intraoral appliance includes flexible, resilient extensions 5040a and 5040b coupled to the corresponding lateral segments 1030a and 1030b at the respective posterior molar portions 1031a, 1031b. The flexible resilient extensions 5040a, 5040b may extend inferior and medial of the lateral segments 1030a, 1030b. The flexible resilient extensions 5040a, 5040b may comprise a patient interface portion 5048 that comprises a relatively lower durometer material.

First electrodes 1050a, 1051a are positioned on a first electrode support 5047a which is coupled to an inferior end 5049a of a first extension 5040a. Second electrodes 1050b, 1051b (FIG. 15) are positioned on a second electrode support 5047b which is coupled to an inferior end 5049b of a second extension 5040b.

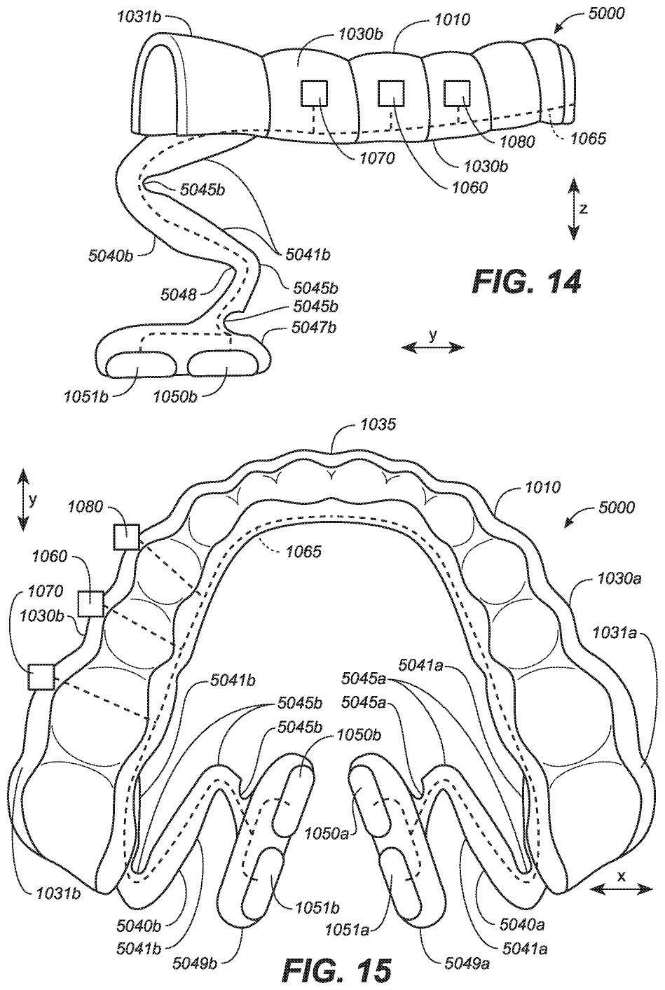

Similar to FIGS. 7-9, the extensions 5040a, 5040b can each comprise a single arm that includes a plurality of more rigid regions 5041a, 5041b joined by more flexible regions 5045a, 5045b (“flexion regions”). However, rather than flexibly linked rigid members, the flexible resilient extensions 5040a, 5040b are formed of a single flexible resilient element with physical features such as thicker regions and/or thinner regions that provide different amounts, degrees and/or directions of flexion along the length of the extension that effectively form segments, areas, locations, regions, portions, points, and/or axes of greater or lesser flexion and varied directions and degrees of flexion. The resilient properties of the element can bias the extensions in a medial/lateral, inferior/superior, and/or anterior/posterior direction, and/or towards an angular orientation, so as to direct or urge the electrodes towards target stimulation tissue, areas or regions. A relatively simple device or manufacturing process (e.g., injection molding) can be used to form the overall shapes of the extensions, possibly including regions having different thicknesses to allow for different degrees of flexibility. More complex (e.g., out of plane) flexion characteristics can be provided, for example, using notches, cut outs and/or other features. Individualized or customized devices can be 3D printed, injection molded, or otherwise constructed. As illustrated in FIG. 14, the less flexible regions 5041a, 5041b do not include additional support or more rigid structures. However, additional support structures or more rigid elements, and resilient elements can be included in some representative embodiments.

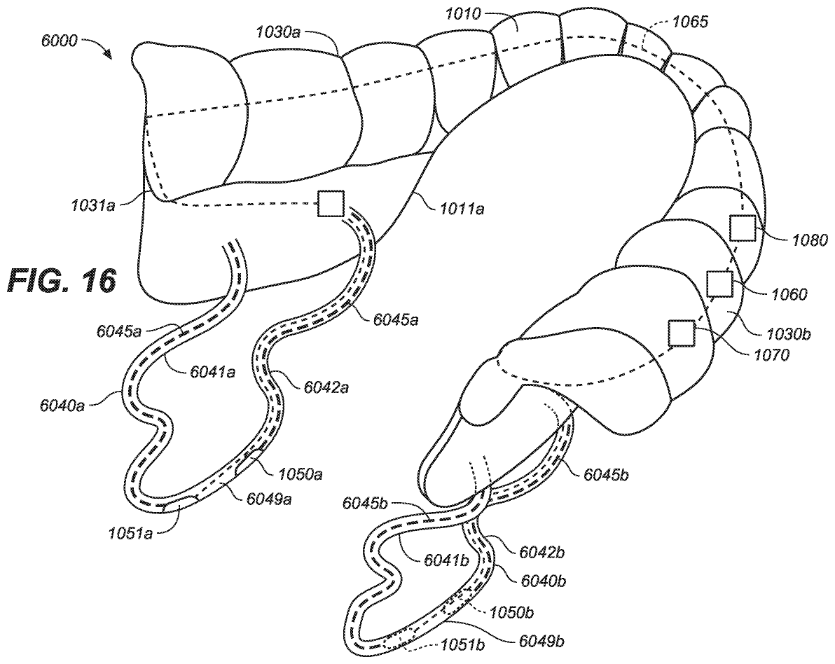

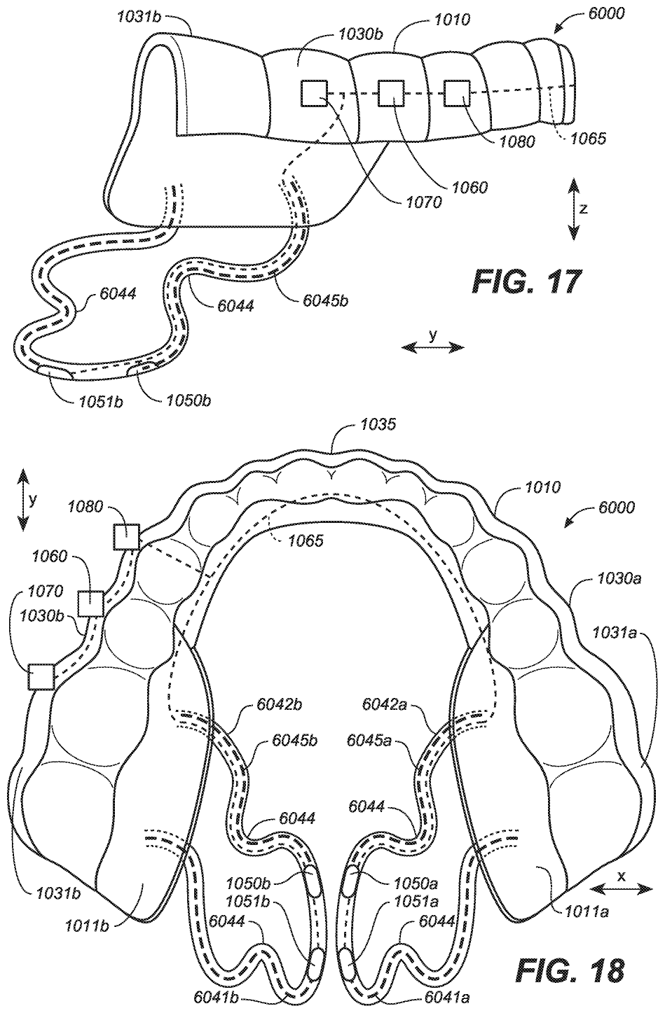

FIGS. 16-19 illustrate another representative removeable intraoral appliance 6000 having an attachment body 1010 and flexible, resilient extensions 6040a and 6040b coupled to corresponding flaps 1011a, 1011b. The flexible resilient extensions 6040a, 6040b may extend inferior and medial of the flaps 1011a, 1011b to limit flexion or movement to locations inferior to the flaps 1011a, 1011b. The extensions 6040a, 6040b can each include at least one corresponding arm 6042a, 6042b coupled to the attachment body flaps at multiple points. The arms 6042a, 6042b can have undulations or bends 6044 along their lengths to provide flexion points, segments, regions, portions, areas, locations, and/or areas that provide stress relief and allow controlled movement, generally as described elsewhere herein. Similar to the arrangement shown in FIGS. 10-12, the arms 6042a, 6042b are thinner and less bulky to provide more comfort to a patient. However, to provide more support along their lengths, the arms 6042a, 6042b do not cross over as they do in FIGS. 10-12.

As shown in FIGS. 16-19, the flexible extensions 6040a, 6040b can each include a corresponding posteriorly originating arm 6041a, 6041b connected to a corresponding anteriorly originating arm 6042a, 6042b. The anteriorly originating arms 6042a, 6042b are coupled to the flaps 1011a, 1011b at a location anterior to where the posteriorly originating arms 6042a, 6042b attach to the flaps 1011a, 1011b. First electrodes 1050a, 1051a are positioned at the inferior end 6049a of the first extension 6040a, and second electrodes 1050b, 1051b are positioned on inferior end 6049b of the second extension 6040b.

The arms 6041a, 6041b, 6042a, 6042b can include a flexible material. The flexible material may also be resilient and/or can comprise wires 6045a, 6045b that act as spring elements or stiffening elements that may be encased in a lower durometer material. The wires 6045a, 6045b can bias the inferior ends 6049a, 6049a in a variety of directions and toward electrical contact with target tissue. The arms 6041a, 6041b, 6042a, 6042b may also operate as tethers or range of motion limiters for the extensions and attached electrodes. The anteriorly and posteriorly originating arms on each side of the appliance 6000 are joined to each other, and, can be formed integrally as a generally U-shaped element, with bends and curves as described above.

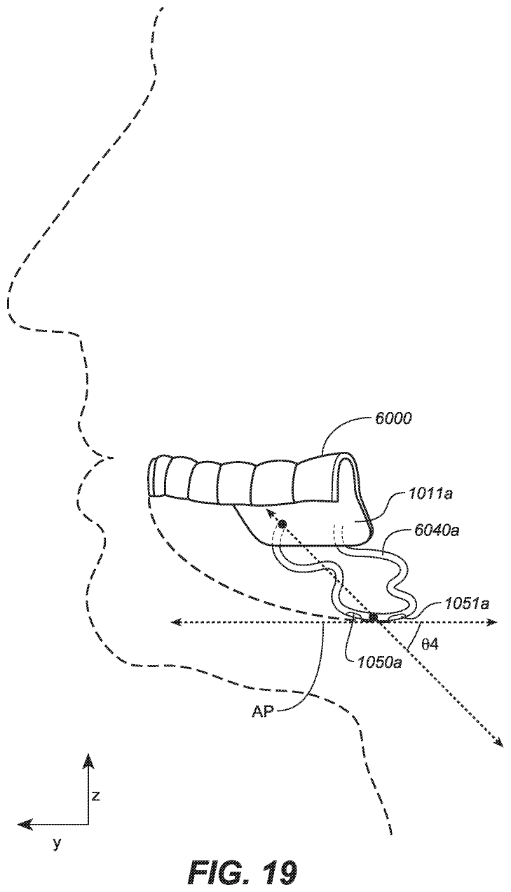

The anteriorly originating arms 6042a, 6042b are attached in anterior positions on the flaps 1011a, 1011b. Referring now to FIG. 19, the anterior attachment points along with the midpoint of the inferior ends 6049a, 6049b define corresponding lines forming angles θ4 with respect to the axial plane AP (as viewed from the y-z plane). The angle is more acute than would be formed by the arrangement shown in FIGS. 10-12. It is expected that the more acute angle will provide greater anterior-to-posterior support of the attached electrodes. The angle θ4 is similar to the angle that would be formed by the anterior arm shown in FIGS. 1-3. However, in the arrangement shown in FIGS. 16-19, the motion of the extensions 6040a, 6040b is limited to areas of soft tissue by the attachment to the flaps 1011a, 1011b as described with reference to FIGS. 10-12, in order to provide greater patient comfort. In representative embodiments, the angle θ4 may range from between 35 degrees and 110 degrees, wherein 0 degrees is in the posterior direction.

FIG. 20 illustrates the intraoral appliance 6000 of FIGS. 16-19 with additional electrodes 1052a, 1053a, 1052b, 1053b located on the arms 6042a, 6042b of the extensions 6040a, 6040b. The electrodes 1050a, 1051a, 1052a, 1053a on extension 6040a and electrodes 1050b, 1051b, 1052b, 1053b on extension 6040b can be selected for activation using any of the techniques described herein. The multiple electrodes 1050a, 1051a, 1052a, 1053a, 1050b, 1051b, 1052b, 1053b can be used to target different areas, anatomical structures, and/or tissue. The electrodes 1050a, 1051a, 1052a, 1053a, and/or 1050b, 1051b, 1052b, 1053b may operate as an array as described herein. In general, in this and other representative embodiments, the different electrode positions on the extensions, and/or an electrode array, can allow the practitioner, patient and/or the device logic to select different current paths and/or directions of current paths through tissue to obtain a desirable stimulation response. Accordingly, prior to deploying the intraoral stimulation device and/or in adjusting a deployed intraoral stimulation device, the stimulation program and electrode selection therein can be tested and updated (e.g., optimized).

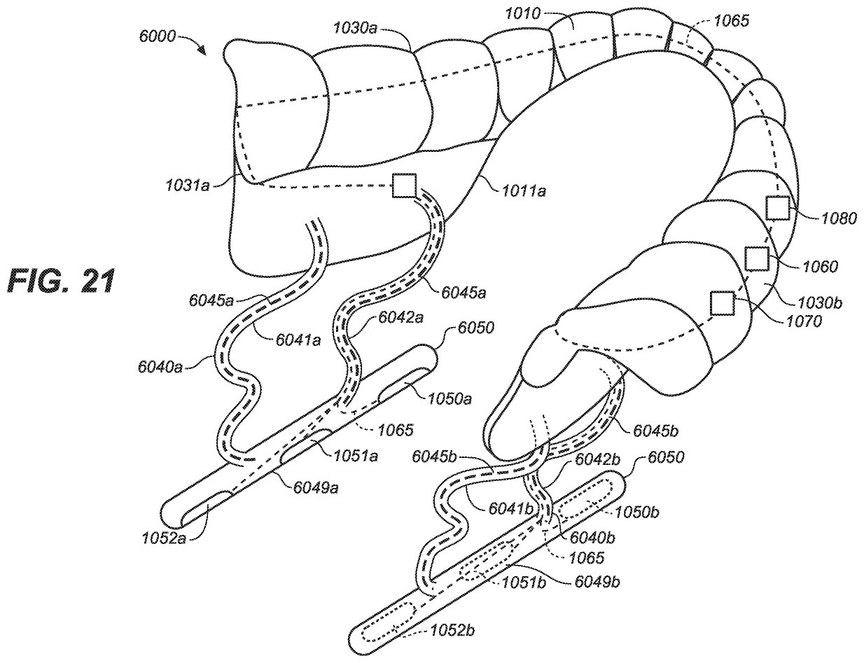

FIG. 21 illustrates the intraoral appliance 6000 generally similar to that shown in FIGS. 16-19, but with each extension 1040a, 1040b further including an elongate bar 6050 positioned at the distal end of the extensions 6040a, 6040b. The bars 6050 extend generally in a posterior-to-anterior direction with electrodes 1050a, 1051a and 1052a positioned along one bar 6050 and electrodes 1050b, 1051b, 1052b positioned on the laterally opposing bar. The electrodes and stimulation parameters can be selected as described herein with respect to FIG. 20. The bars 6050 in some representative embodiments can be more flexible, more rigid, or may exhibit generally similar material properties as the arms 6042a, 6042b. More flexible bars can allow the bars to conform to the tissue. The elongate bars can provide stimulation options particularly when the tongue moves (which tends to move the extensions and electrodes posteriorly and anteriorly). The electrodes 1050a, 1051a, 1052a, and/or 1050b, 1051b, 1052b, may operate as an array as described in more detail herein.

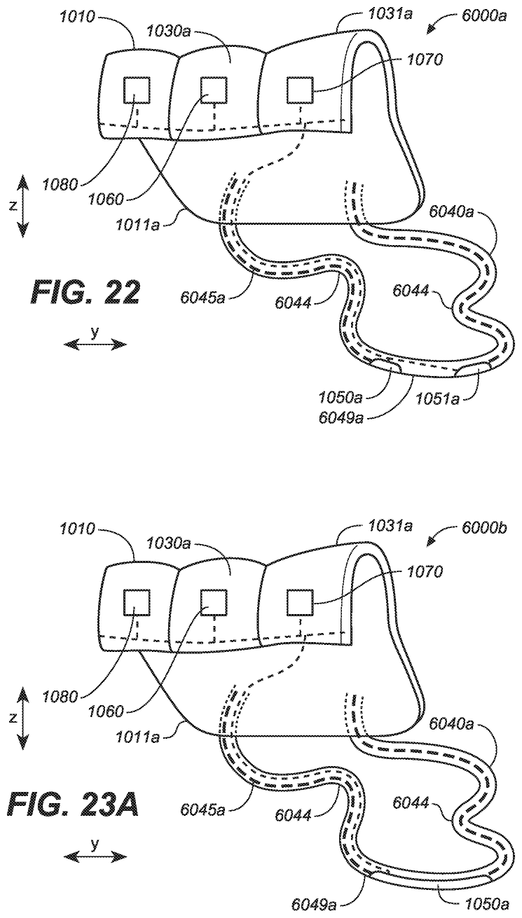

FIG. 22 illustrates a representative removeable intraoral appliance 6000a having a single attachment body 1010 and a flap 1011a with a single attached extension 6040a and electrodes 1050a, 1051a. The intraoral appliance attachment body 1010 attaches to one side of the patient's oral cavity and has one extension instead of two extensions on laterally opposite sides of the oral cavity. The appliance 6000a may be used to provide electrical stimulation to one side of the oral cavity by directing current between electrodes 1050a, 1051a.

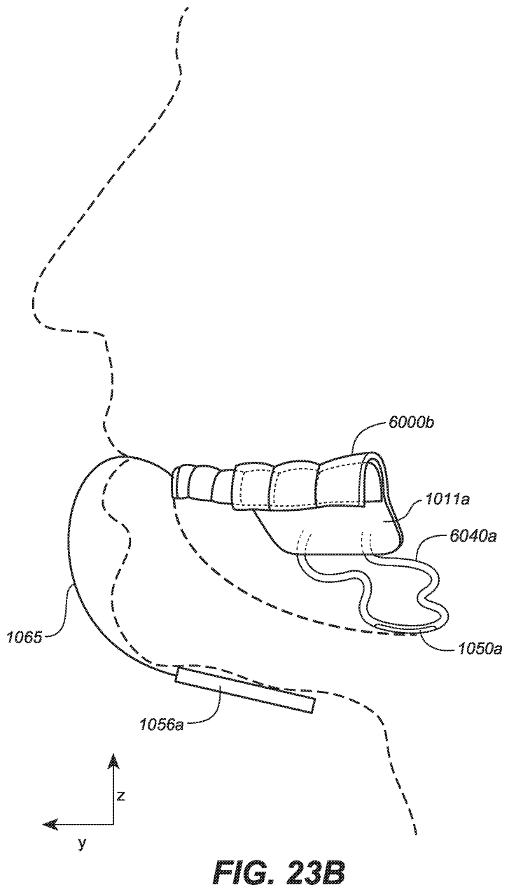

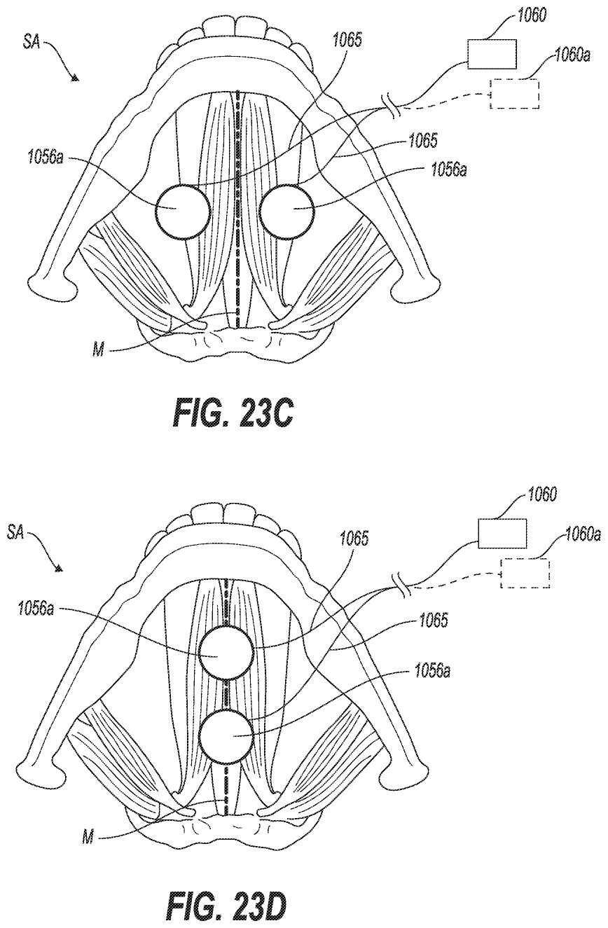

FIGS. 23A-23B illustrate another representative removeable intraoral appliance 6000b having a single attachment body 1010 and a flap 1011a with a single attached extension 6040a and one or more first electrodes 1050a. The intraoral appliance attachment body 1010 attaches to one side of an oral cavity and has one extension instead of two extensions on laterally opposite sides of the oral cavity. A second (opposing) electrode 1052a may be coupled to a location external to the oral cavity, as shown in FIG. 23B. The appliance 6000b can be used to provide electrical stimulation to the tissue of the oral cavity by directing current between the first and second electrodes 1050a, 1052a. The second electrode 1052a can be coupled to the electronic circuitry with a connector or wire 1065. By changing the position of the (external) electrode 1052a, the practitioner and/or patient can controllably direct the electrical current to the target tissue.

FIG. 23B illustrates a single external electrode 1056a (e.g., a submental electrode) positioned on the patient's skin, e.g., under the chin, in combination with an appliance having a single extension 6040a. In other embodiments, the overall system can include more than one external electrode, in combination with an intraoral appliance that includes more than one (e.g., two) extensions, carrying any suitable number of intraoral first electrodes. For example, FIGS. 23C-23D illustrate removeable extraoral electrodes 1056a (e.g., submental electrodes) placed on the surface of the skin in the submental area SA. For example, with reference to FIG. 29, the external electrodes can be positioned under the chin in the muscular area between the hyoid bone HB and the mandible Ma. The external electrodes 1056a can be used in conjunction with any of the appliances described herein. The external electrodes 1056a can transmit electrical energy to and/or from a corresponding first (intraoral) electrode through the genioglossus GG and geniohyoid GH. In some other embodiments, one external electrode 1056a can transmit electrical energy to and/or from a second external electrode 1056a. Two or more external electrodes 1056a can be placed laterally opposite each other (e.g., to the left and right of the midline Mi of the submental area SA) as shown in FIG. 23C, or in an anterior-posterior orientation (e.g., on the midline Mi). Suitable materials for the external electrodes 1056a include stainless steel, titanium, platinum, platinum iridium, and other conductive materials. The external electrodes 1056a may be or include a solid piece of conductive material, a flexible mesh of conductive material, conductive polymers, conductive hydrogels, and/or flexible pieces of conductive foil. While FIGS. 23C-23D illustrate two circular-shaped external electrodes 1056a, the external electrodes 1056a can have any of a variety of other suitable shapes and/or numbers, and/or can be placed in other suitable locations. For example, the system can include between one and six external electrodes 1056a. Suitable shapes for the external electrodes 1056a can include square, rectangular, rectilinear, elliptical, oval, and/or irregular. The external electrodes 1056a can be placed on the midline Mi, on either side of the midline Mi, and/or on both sides of the midline Mi.

In some embodiments, the external electrodes 1056a are electrically coupled (e.g., via wires 1065) to an internal pulse generator which forms part of the electronics circuit 1060 shown in FIG. 23A. For example, a power generating unit (i.e., a power source) can be integrated into an appliance located on the mandibular teeth and/or maxillary teeth. In other embodiments, the external electrodes 1056a can be energized by the internal pulse generator via electromagnetic coupling (e.g., inductive, capacitive, and/or RF coupling). In other embodiments, the external electrodes 1056a can be electrically coupled to a small (e.g., external wearable) pulse generator 1060a (shown in dashed lines) by way of the wires 1065 or another suitable electrical connection. In other embodiments, the external electrodes 1056a can be energized by the external pulse generator 1060a and/or another suitable energy source via electromagnetic coupling (e.g., inductive, capacitive, and/or RF coupling). The intraoral electrodes 1050a can be energized by the external pulse generator 1060a and/or another suitable energy source via electromagnetic coupling (e.g., inductive, capacitive, and/or RF coupling). For example, the power source can be placed in the submental area SA, such as in proximity to the hypoglossal nerve HGN (FIG. 29D). In a further example of this embodiment, the external electrodes 1056a and the power generating unit can be combined into one component.

The external electrodes 1056a can be adhered to the skin in a fashion similar to that used for transcutaneous electrical nerve stimulation (TENS) electrodes. A glue and/or double-sided medical tape can be used to adhere the submental electrodes 1056a to the skin located in the submental area SA. Additionally, a preparatory material such as a wipe or coupling gel may be applied to the skin to clean the surface and/or improve adhesion. In a representative embodiment, the glue and/or tape keeps the external electrodes 1056a bonded to the skin for approximately 10-12 hours. Suitable materials for the glue can include cyanoacrylates and polyurethane glues, as well as adhesive hydrogels. The patient can use mechanical force (e.g., peeling) and/or a dissolving solvent to remove the submental electrodes 1056a from the skin after use, e.g., once per day.

Depending on the embodiment, electrical currents can be directed in any of a number of suitable combinations. For example, one or more electrical currents can be directed between one or more intraoral electrodes and another one or more intraoral electrodes. In another embodiment, one or more electrical currents can be directed between one or more intraoral electrodes and one or more external electrode. In another embodiment, one or more electrical currents can be directed between one or more external electrodes and another one or more external electrodes. In another embodiment, one or more electrical currents can be directed between one or more intraoral electrodes and another one or more intraoral electrode, in combination with one or more electrical currents directed between one or more intraoral electrodes and one or more external electrodes. In another embodiment one or more electrical currents can be directed between one or more intraoral electrodes and another one or more intraoral electrodes, in combination with one or more electrical currents directed between one or more external electrodes and another one or more external electrodes. In any of these embodiments, the currents can be directed in a predominantly lateral direction, a predominantly first axial direction (e.g., along an anterior-posterior axis), and/or a predominantly second axial direction (e.g., along a superior-inferior axis). The particular current direction(s) can be selected by pairing different electrodes, and can be tailored to individual patient preferences and/or efficacy.

An advantage of embodiments that include external electrodes is that such electrodes can increase the probability of stimulating the genioglossus and/or geniohyoid muscles, while remaining non-invasive. Depending on patient preferences and/or efficacy, the external electrodes can be used to generate transcutaneous currents in addition to, or in lieu of, the intraoral currents (e.g., lateral currents) described above.

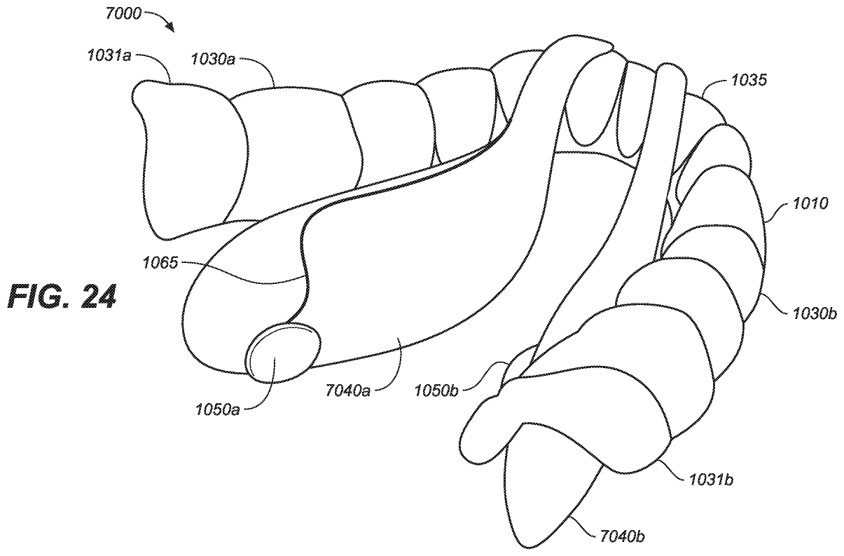

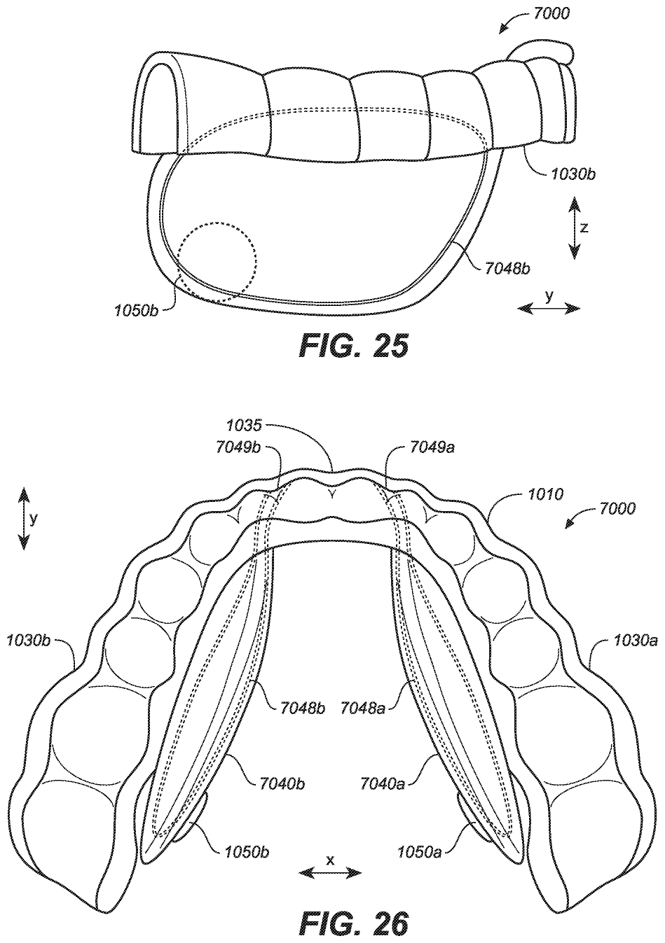

FIGS. 24-26 illustrate another representative removeable intraoral appliance 7000 configured in accordance with embodiments of the present technology. The appliance 7000 includes electrodes 1050a, 1050b coupled to corresponding inflatable flexible resilient extensions 7040a, 7040b which are then respectively coupled to the medial or anterior portion 1035 of the attachment body 1010. The flexible resilient extensions 7040a, 7040b can alternatively be coupled to the lateral segments 1030a, 1030b at a posterior or other location. The electrodes 1050a, 1050b can be coupled to the extensions 7040a, 7040b at an inferior end of the extensions. Referring to FIG. 26, the flexible resilient extensions 7040a, 7040b can include bladders 7048a, 7048b and valves 7049a, 7049b for receiving a fluid inflation medium (e.g., air or a liquid or polymer, such as a polymer that cures to or forms a malleable structure). The bladders 7048a, 7048b can be inflated to provide a flexible resilient electrode support that positions the electrodes 1050a, 1050b at corresponding target locations. The flexible resilient extensions 7040a, 7040b can have a shape that further facilitates positioning the electrodes medial to sublingual and/or other salivary glands.

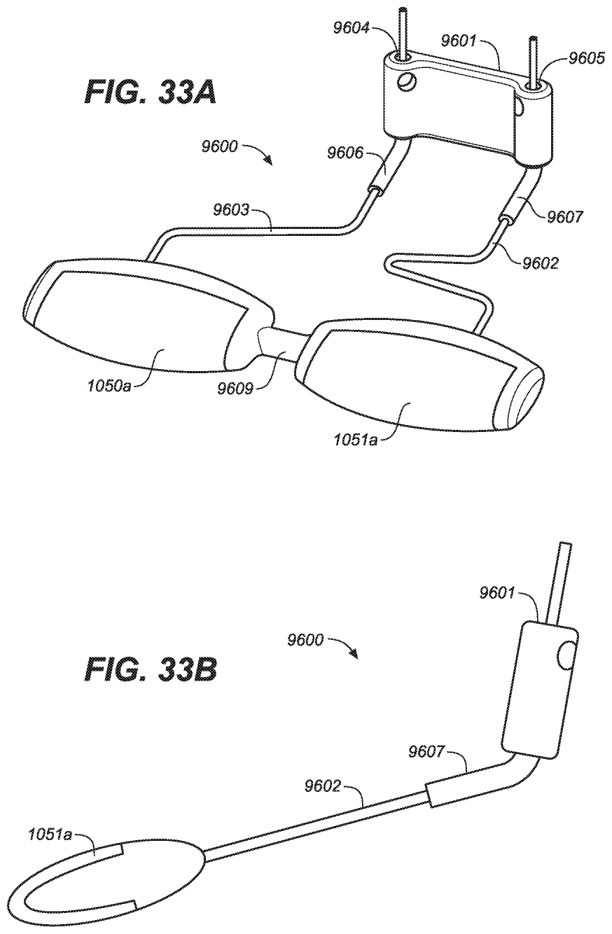

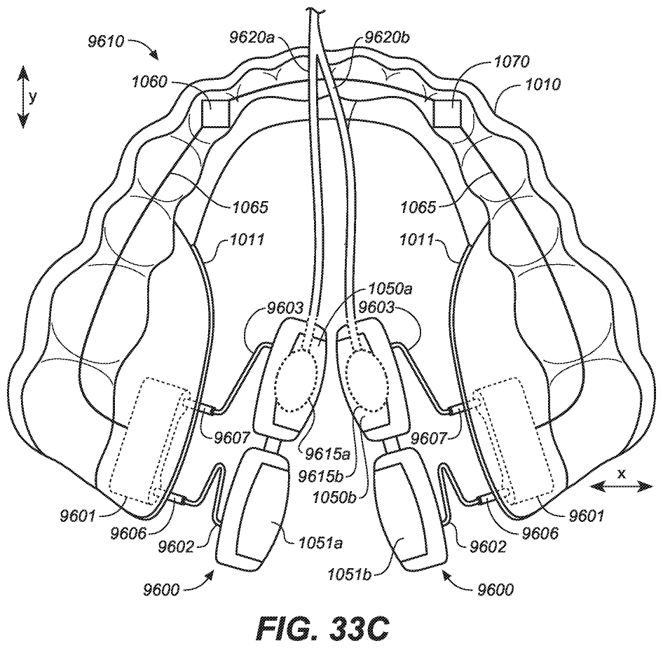

The inflation level can be adjusted for an individual patient to provide comfort and proper electrode positioning, and, can allow anatomical movement while at the same time providing support and proper fit. While the electrical stimulation can be generally as described herein, the inflatable elements of the extensions can reduce undesirable movement by the extension elements while maintaining some flexibility. The inflatable elements can also provide patient comfort due to the conformability of the extensions to an individual patient's oral cavity. The bladder or bladders can also be inflated, for example, during testing to ensure good electrode contact during evaluation. The bladders can also be used as sensors that sense pressure. Pressure changes can indicate a response to stimulation, for example by tongue movement. Pressure changes can also indicate patient position. For example, a differential pressure between connected bladders on opposing lateral sides can indicate patient movement or position. The bladder may form an entire extension or may be positioned on a portion of an extension. One or more bladders or inflatable elements can be positioned on extensions including, but not limited to, the extensions illustrated in FIGS. 1-33C. FIGS. 33A-C (described in further detail later) also illustrates a bladder positioned on a portion of an extension.

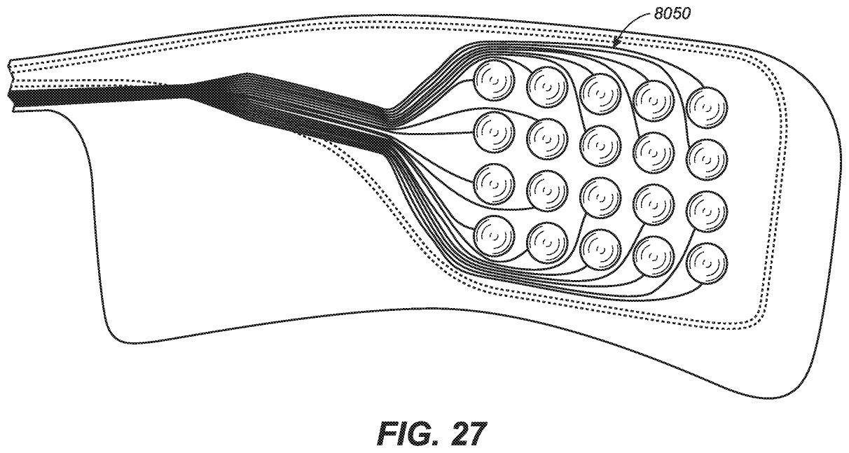

FIG. 27 illustrates an array of electrodes 8050 that may be used instead of any single electrode or electrode pair described in FIGS. 1-26 herein (e.g., electrodes 1050a, 1050b, 1051a, 1051b). The electrodes of the array 8050 are coupled to the electronic circuitry 1060 (FIG. 1), which may include a program or controller logic to selectively activate any one or more of these electrodes at an anodic or cathodic current or voltage, or any electrode can operate as one of a multipolar electrode arrangement. An opposing electrode array can be positioned on an opposite lateral side of the patient's oral cavity, with individual electrodes forming one or more electrode pairs with the opposing array. Electrodes and electrode pairs can be selected based on feedback from one or more sensors 1070 (FIG. 1) that indicate the effectiveness of the stimulation. One or more of the electrodes in the array 8050 can be used to sense the patient's response to electrical stimulation, in additional to or in lieu of delivering the stimulation. Electrodes can also be selected in view of changes in the patient's response to electrical stimulation (e.g., during sleep) due to movement of the electrodes, and/or other factors (e.g., habituation).

FIG. 28A is a partially schematic, cut-away coronal view of a patient illustrating first and second electrodes 1050a, 1050b positioned in the patient's oral cavity. Extension member 4040b is shown as a representative example, extending medially and inferior to the flap 1011b of the body 1010 and positioned adjacent a sublingual salivary gland. Other representative extension members can be similarly positioned in a patient's oral cavity. The second electrode 1050b is shown oriented at an initial angle θ1 with respect to a sagittal plane SP1 which is perpendicular to the coronal plane. The angular rotation of the electrode (about the y axis) with respect to the sagittal plane is referred to herein as the roll. Any of the foregoing representative oral appliances shown in FIGS. 1-26 can be configured to orient the electrode(s) at a predetermined roll angle or within a range of predetermined roll angles in order to direct the electrodes, or current from the electrodes, toward the target stimulation tissue, areas and/or regions. An “initial” angle θ1 as used herein is evident when the oral appliance is not yet positioned in the patient's oral cavity. The value of the angle may change once the appliance is actually positioned in an oral cavity, for example due to an individual patient's particular anatomy. However, the electrode will continue to be biased toward the “initial angle”. When positioned in the patient's oral cavity, the flexible extension of the appliance can flexibly permit the electrode to roll. The extension member can also be resilient, causing the electrode to tend to move toward the initial angle θ1. The extension member can include struts, wires, springs, material thickness variations, supports, reinforcements with stiffer materials and/or other features that limit the amount of roll. The initial angle θ1 can be from about 5 degrees to about 90 degrees with respect to the sagittal or x-z plane (with 0 degrees being in the inferior direction), or between 25 and 75 degrees with respect to the sagittal or x-z plane, or between 25 and 50 degrees with respect to the sagittal or x-z plane. According to some embodiments herein, the initial angle θ1 of the electrode is selected based on an individual patient's anatomy and/or response to the electrical stimulation. The amount of roll flexibility, e.g., the degree to which the extension will allow an electrode to roll away from the initial angle, can also be controlled or preset. For example, in some representative embodiments, the roll flexibility can be up to 45 degrees from the initial angle, in either direction.

FIG. 28B is a partially schematic, cut-away sagittal view of the patient illustrating the first electrode 1050a oriented at an initial angle θ2 with respect to a transverse plane AP which is perpendicular to the sagittal plane. The angular rotation of the electrode (about the x axis) with respect to the axial plane is referred to herein as the pitch. The oral appliance 1000 can be configured to bias the first electrode 1050a toward an initial pitch angle in order to direct the electrode (or current from the electrode), toward the target stimulation tissue, areas or regions. According to some representative embodiments, the initial pitch angle can be between 0 and 45 degrees. The amount of pitch flexibility, i.e., the amount the extension will allow an electrode to pitch from the initial angle can also be controlled or preset. For example, in some representative embodiments, the pitch flexibility can be up to 45 degrees from the initial angle, in either direction.

FIG. 28C is a partially schematic, cut-away transverse view of the patient depicting the first and second electrodes positioned in the oral cavity. The second electrode 1050b is shown oriented at an initial angle θ3 with respect to a coronal plane CP which is perpendicular to the axial plane. The angular rotation of the electrode (about the z axis) with respect to the coronal plane is referred herein to as the yaw. The corresponding oral appliance can be configured to bias the electrode toward an initial yaw angle in order to direct the electrodes (or current from the electrode), towards target stimulation tissue, areas or regions. According to some representative embodiments the initial yaw angle can be between 0 and 45 degrees. The amount of yaw flexibility, i.e., the amount the extension will allow an electrode to yaw from the initial angle can also be controlled or preset. For example, in some representative embodiments, the yaw flexibility can be up to 45 degrees from the initial angle, in either direction.

Any of the electrodes described herein can have an angular orientation defined by one or more of the roll, pitch and/or yaw angles. The specific angle or combination of angles can be preset to fit a large portion of the patient populations or can be tailored to a specific patient.

FIGS. 30A and 30B illustrate a representative example in which an intraoral appliance having any one or combination of the features described herein with reference to FIGS. 1-33C positions any of the electrodes described herein within the sublingual sulcus near the nerve branches that activate the geniohyoid muscle GH and the genioglossus horizontal muscle GGh. Several of the physiological features shown in FIG. 30A are also shown in FIG. 29A, discussed previously.

As an example, a representative electrode 1050a is coupled to a representative extension 4040a (FIG. 30B) as positioned medial with respect to the sublingual salivary gland and the submandibular salivary gland. The flexible resilient extension 4040a of the intraoral appliance shown in FIG. 30B extends medially of the attachment body 1010 to constrict and/or bias the electrode 1050a to a position within the sublingual sulcus. The flexible resilient extension 4040a positions the electrode 1050a in a manner that facilitates the electrode seating itself within the sublingual sulcus. The flexible resilient extension 4040a permits the electrode 1050a to move when the tissue or tongue move, for example, because of a change in the patient position, sleep state and/or tongue and/or muscle position in the upper airway. A second electrode or electrode array (not shown in FIG. 30B) can be positioned in the laterally opposing sublingual sulcus and current can be directed between the electrodes on opposing sides of the tongue.

FIG. 30B also shows a line L1 about at which the mylohyoid muscle attaches to the mylohyoid ridge of the mandible. Above the line L1, where relatively less soft tissue movement occurs, the rigid flap 1011a can prevent significant movement of the attached extension member 4040a. Below the line L1, is a region of soft tissue in which the extension 4040a and attached electrode 1050a are permitted greater range of motion. Accordingly, in order to improve electrode/tissue contact and patient comfort, the motion of the flexible components of the extension members can be limited to the areas of soft tissue. Further, the motion of the flexible connectors can be limited to the location where strain relief from movement is desired. This region of tissue movement is generally below the mandible.

While FIGS. 30A-30B illustrate a particular potential stimulation target, there are a number of other targets within the oral cavity that, when stimulated, will potentially prevent or reduce collapse or obstruction of the upper airway. Accordingly, an oral appliance as described herein can position one or more electrodes to target a variety of anatomical and/or nerve structures of the upper airway/oral cavity to improve upper airway patency or tone.

The electronics circuit described herein can include a signal generator (e.g., a pulse generator) that is powered by a rechargeable battery and that can deliver stimulation pulses, such as a biphasic waveform. The stimulation pulses may be symmetrical or asymmetrical, depending on the application. The stimulation waveform can be adjustable within a range of waveforms, via the electronic circuitry. Such adjustments may be based on sensed feedback. The stimulation may occur continuously or periodically (e.g., in accordance with a duty cycle) and/or can be triggered by a sensed parameter.

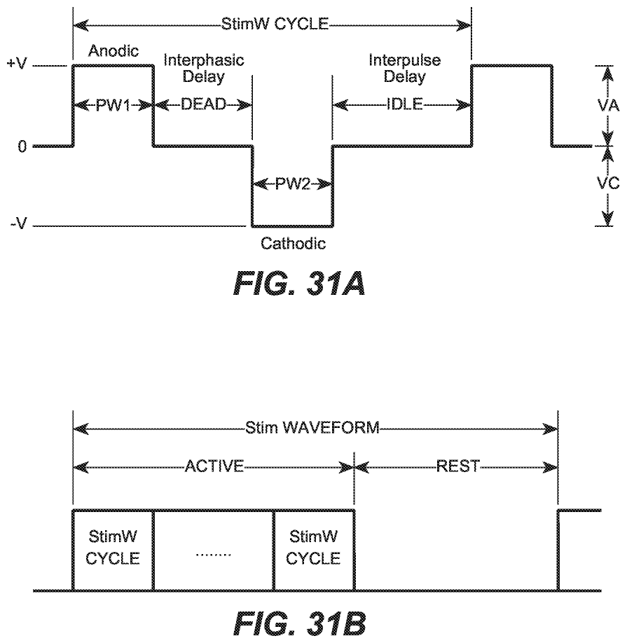

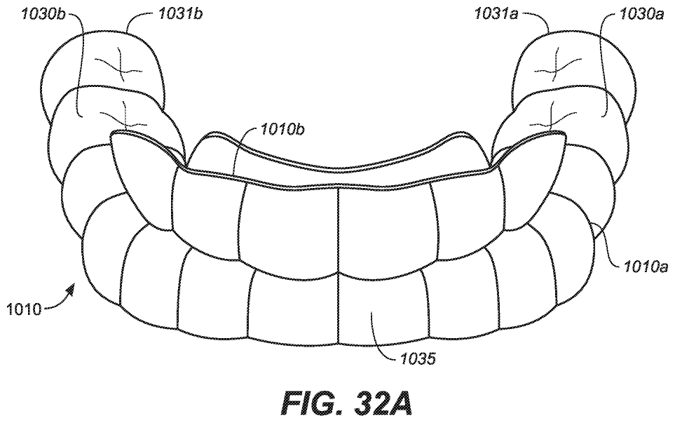

A variety of suitable electrical stimulation waveforms and techniques can be used to stimulate the patient's tissue. Representative examples are illustrated in FIGS. 31A and 31B and include a series of a biphasic stimulation pulses or stimulation wave cycles (identified as StimW CYCLE). The stimulation waveform parameters can include active cycles and rest cycles. The active cycles include one or more stimulation wave cycles. The stimulation wave cycle shown in FIG. 31A comprises an anodic pulse followed by an interphasic delay, a cathodic pulse and then an interpulse delay. Accordingly, a stimulation wave cycle includes the following parameters: anodic pulse width (PW1), anodic amplitude (e.g., voltage or current amplitude VA), interphasic delay/dead time, cathodic pulse width (PW2), cathodic amplitude (e.g., voltage or current amplitude VC), interpulse delay/idle time, and peak-to-peak amplitude (PP). The parameters may also include identity of the electrode(s) to which the signal is directed. The anodic pulse width (PW1) in some representative embodiments is between 40 μs and 400 μs. The anodic amplitude (VA) in some representative embodiments ranges from 0.3 V to 50 V. The interphasic delay in some representative embodiments can be from 10 μs to 100 μs. The cathodic pulse width (PW1) is some representative embodiments is between 40 μs and 400 μs. The cathodic amplitude (VA) in some representative embodiments ranges from 0.3 V to 50 V. The interpulse delay in some representative embodiments can be from 10 μs to 100 μs. The peak-to-peak amplitude in some representative embodiments can be from about 5 mAmp to 30 mAmp.

FIG. 31B illustrates a stimulation waveform comprising an active cycle and a rest cycle. The active cycle includes one or more of stimulation cycles (StimW CYCLE) as shown in FIG. 31A. The rest cycle has no stimulation cycles. According to some representative embodiments, the ratio of active duration to rest duration can be between 1:1 and 1:49. As a representative example, if the ratio is 1:9, and there are 300 active cycles, there can be 2700 rest cycles.

In a representative example the stimulation voltage may be presented independently to each contact or electrode. For the positive pulse, the positive contact can be pulled to the drive voltage and the negative contact is pulled to ground. For the negative pulse, the negative contact can be pulled to the drive voltage and the positive contact is pulled to ground. For dead time and idle time, both contacts are driven to ground. For the rest time, both contacts are at a high impedance. To prevent DC current in the contacts, each half-bridge can be coupled to the contact through a capacitor, for example, a 100 capacitor. In addition, a resistor can be placed in series with each capacitor to limit the current in the case of a shorted contact. The pulses of the therapeutic waveform cycle may or may not be symmetric, but, are generally shaped to provide a net-zero charge across the contacts.

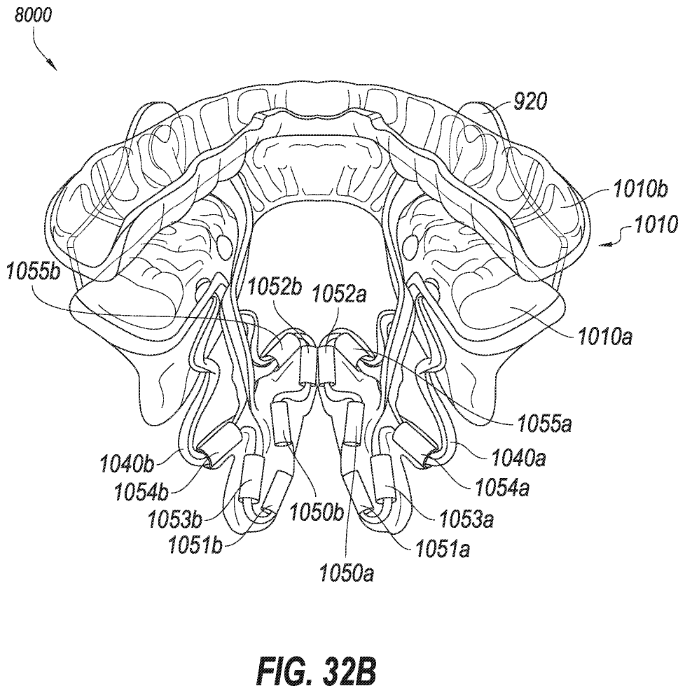

FIG. 32A illustrates a body 1010 of an oral appliance that may be similar to the oral appliance of any of the foregoing representative embodiments. The overall body 1010 can include a lower body 1010a and an upper body 1010b coupled to the lower body 1010a. The upper body 1010b is configured to secure to one or more of the upper teeth of the upper jaw so that when the appliance is positioned in an oral cavity, the upper jaw and lower jaw are generally more stationary with respect to each other. This additional attachment to the upper teeth provides a more stable electrode connection to the tissue of the oral cavity by preventing extra movement of the upper and lower jaws with respect to each other. The attachment can be flexible or rigid. The body 1010 can be configured to engage any suitable combination of upper and lower teeth, including front teeth and/or molars.

FIG. 32B illustrates another representative removeable intraoral appliance 8000 configured in accordance with embodiments of the present technology. The appliance 8000 includes electrodes 1050a, 1051a, 1052a, 1053a, 1054a, 1055a, 1050b, 1051b, 1052b, 1053b, 1054b, 1055b coupled to corresponding flexible resilient extensions 1040a, 1040b which are coupled to the attachment body 1010. The attachment body 1010 can include a lower body 1010a, an upper body 1010b, and one or more elastomeric attachment elements 920 that connect the upper body 1010b to the lower body 1010a. Along with the upper body 1010b, the elastomeric attachment elements 920 can further prevent unwanted movement of the lower jaw relative to the upper jaw to provide a more stable electrical connection to the tissue of the oral cavity. The elastomeric attachment elements 920 can be constructed from a variety of suitable materials, including polyurethane, silicone, natural rubber, thermoplastic elastomers, etc. The appliance 8000 may be used to provide electrical stimulation to the oral cavity by directing one or more electrical currents between any of a number of suitable combinations of the electrodes 1050a, 1051a, 1052a, 1053a, 1054a, 1055a, 1050b, 1051b, 1052b, 1053b, 1054b, 1055b. In any of these embodiments, the currents can be directed in a predominantly lateral direction, a predominantly first axial direction (e.g., along an anterior-posterior axis), and/or a predominantly second axial direction (e.g., along a superior-inferior axis). In a representative embodiment, the currents can be directed in the predominantly second axial direction (e.g., along the superior-inferior axis). The term “predominantly” when used in connection with a direction refers to a vector having a component in one direction that is greater than the components in the other two directions. While FIG. 32B illustrates the lower body 1010a having six electrodes, and the upper body 1010b having no electrodes, the upper body 1010b can include one or more electrodes in other embodiments, and/or the lower body 1010a can include numbers of electrodes other than six.

Intraoral stimulation devices configured in accordance with embodiments of the present technology can have moveable electrodes that can be moved to various positions so that when placed in a patient's oral cavity they can be different locations. Thus, the electrodes can be positioned in different locations to obtain a desired response and identify a desired electrode position (configuration). Each individual patient's anatomy is different, and a more effective stimulation response, can be obtained for an individual patient by identifying positions of and locations for electrode and placing electrodes in these desired positions and locations. The intraoral stimulation devices can have moveable electrodes that can be moved in one or more directions. For example, the electrodes can be moved in anterior-posterior, inferior-superior, and/or medial-lateral directions. In addition to or in lieu of axial motion, the electrode(s) can be moved to different angular orientations.

According to some representative embodiments, a nasal endoscope can be used to observe the upper airway response of at least some anatomical structures, for example, tissue tensing and bulk movement. The anatomical structures observed can include, but are not limited to: the velum, oropharynx, epiglottis and/or tongue base. The response can be scored by an observer and the results used to select a final extension member and/or electrode configuration. Alternatively, or in addition, sensors can be used to sense response to stimulation as described herein as well as measurements in a sleep lab or similar environment where effectiveness can be evaluation, for example using an AHI.

FIGS. 33A-33B illustrate an adjustable extension 9600 that can be adjusted or customized for a particular patient. FIG. 33A is an isometric illustration of the extension 9600 and FIG. 33B illustrates a side view of the anterior portion of the extension. FIG. 33C illustrates an intraoral appliance with extensions 9600 installed. The extension 9600 can be used in an intraoral stimulation device and/or a test device. The extension 9600 can be coupled to a mouthpiece or attachment body 1010 (FIG. 33C) and positioned on a lateral side of the patient's oral cavity. An opposing extension 9600 can be positioned on an opposite lateral side of a patient's oral cavity and attached to the attachment body 1010. Each extension 9600 can be separately adjusted and customized for a particular patient. The laterally opposing extensions can be symmetric or different from each other, depending for example, on patient physiology.

Referring now to FIG. 33A, the extension 9600 comprises a mounting base 9601 that is couplable to an attachment body. A posterior flexible resilient wire 9602 extends through a posterior opening 9604 in the mounting base 9601 and then through an angled tube 9606. A first electrode segment 1050a is attached at the distal end of the wire 9602. An anterior flexible resilient wire 9603 extends through an anterior opening 9605 in the mounting base 9601 and then through an angled tube 9607. A second electrode segment 1051a is attached at the distal end of the wire 9603. The electrode segments 1050a, 1051a can be generally flat, or can have other suitable shapes. The electrode segments 1050a, 1051a can extend in generally posterior to anterior directions. The electrode segments 1050a, 1051a are partially encased in a flexible material and linked by a flexible link 9609. The wires 9602, 9603 moveably extend or retract through the angled tubes 9606, 9607 to direct the wires 9602, 9603 in superior-inferior and medial-lateral directions to position the electrodes 1050a, 1051a different locations. Each wire 9602, 9603 can also be separately extended or retracted to vary the electrode locations. The angled tubes 9606 can be made of a deformable material, such as stainless steel. Accordingly, the extent to and/or the direction in which the tubes bend can be independently adjusted to adjust the angles of the wires and attached electrodes. The electrodes 1050a, 1051a are electrically connected to electronic circuitry 1060 (not shown in FIG. 33A). The patient's response to electrical stimulation can be observed with the electrodes at or in any of a variety of locations and positions to identify desired electrode positions. Once a desired position is identified, the wires can be fixed, for example using set screws, an adhesive, wire crimping and/or other suitable devices. The identified desired extension and electrode positions can also be used to manufacture a customized device for the patient.

In some representative embodiments, the electrodes can be generally flat to provide a greater surface area for tissue contact. While the electrodes are shown as being flat in this particular embodiment, in representative embodiments, other electrode shapes or configurations may be used, including for multiple electrode configurations. The flexible link 9609 between the electrodes can allow the flexibly linked electrodes 1050a, 1051a to conform to adjacent tissue and can permit some independent adjustment of the wires. The electrode placement may also be readjusted after use in the event the desired stimulation location shifts or if other changes are desired to improve comfort and/or performance.

In FIG. 33C, two extensions 9600 are illustrated attached to corresponding wings 1011 on the body 1010 of a representative appliance 9610. Two electrodes 1050a, 1051a are positioned on one extension 9600 so as to laterally oppose two corresponding electrodes 1050b, 1051b on the other extension 9600. In addition, one or more inflatable members (e.g., bladders) 9615a, 9615b are shown located at lateral locations on the extensions 9600. The inflatable bladders 9615a, 9615b can be used to position the electrodes medially, to provide greater electrode contact with the adjacent tissue, to create comfort, and/or to be used as pressure sensors. The inflatable bladders 9615a, 9615b are connected to inflation lines 9020a, 9020b which connect to a source for an inflation medium (not shown). If used as a pressure sensor, the sensors are coupled to the electronics 1060 by way of connectors 1065. The pressure sensors can also sense patient tongue movement which may be indicative of the effectiveness of the electrical stimulation. The inflation lines 9020a, 9020b may or may not be connected or linked. For example, if the lines 9020a, 9020b are independent, they can independently position the electrodes on each side and they can independently sense pressure (e.g., by being coupled to a pressure sensor). If the lines are connected, they can shift the inflation medium from one side to the other, for example, when a patient shifts position (or shifts tongue position), thus improving electrode contact on the side where the patient position where needed. If the lines are connected, a differential pressure between the bladders can indicate patient position or respiration characteristics. Inflatable bladders can be added to any of the extensions described in representative embodiments herein. Inflatable bladders may be added to any location on the intraoral stimulation device to improve electrode, appliance body positioning and or patient comfort. While two inflatable bladders are shown in FIG. 33C, any suitable number of bladders can be used in the appliance 9610, depending upon the particular application. For example, the appliance 9610 can include one inflatable bladder per electrode and/or one inflatable bladder per multiple electrodes.

In many operations, testing and use of the intraoral stimulation device can be performed by a practitioner, a patient, and/or may be automated or semi-automated, e.g., using software and/or computer logic.

In the foregoing description, numerous specific details are set forth to provide a thorough understanding of the present technology. In the foregoing description and for purposes of explanation, specific nomenclature is set forth to provide a thorough understanding of the present technology. However, it will be apparent to one skilled in the art that these specific details may not be required to practice the present technology. In other instances, well-known circuits and devices are shown in block diagram form to avoid obscuring aspects of the present disclosure. The term “coupled” as used herein means connected directly to or connected through one or more intervening components, circuits, or physiological matter. Any of the signals provided over various buses described herein may be time-multiplexed with other signals and provided over one or more common buses. Additionally, the interconnection between circuit elements or software blocks may be shown as buses or as single signal lines. Each of the buses may alternatively be a single signal line, and each of the single signal lines may alternatively be a bus, and a single line or bus might represent any one or more of a myriad of suitable physical or logical mechanisms for communication between components. Further, the logic levels and timing assigned to various signals in the description below are arbitrary and/or approximate, and therefore may be modified (e.g., polarity reversed, timing modified, etc.) as desired.

Elements from embodiments disclosed herein may be included in or substituted into other representative embodiments and/or may be combined with different illustrated representative embodiments in any suitable manner. For example, the feature of representative extensions attached to the lateral segments of the appliance by way of rigid flaps as shown in FIGS. 7-12 and 16-21B may be used in combination with elements of other extension members and oral appliances. FIGS. 1-6, 22-24 and illustrate single electrodes on each lateral side of the oral appliance while FIGS. 7-12, 16-21B, 25, and 33A-33C show multiple electrodes. Single electrodes on lateral sides or multiple electrodes lateral sides may be used in various representative embodiments including those illustrated herein. While FIGS. 7-9 and 19-23B illustrate extensions with single extension attachment points to the appliance, in representative embodiments, either single or multiple attachment points may be used to attach an extension to the body of the oral appliance. Rigid elements may or may not be incorporated into extensions or extension arms of representative embodiments. Springs or resilient members may or may not be used in representative embodiments. Undulating or sinusoidal features may or may not be used in representative embodiments. Thicker inferior ends of extensions may or may not be included in representative embodiments. In representative embodiments, arms of extensions may or may not cross over each other. In representative embodiments, an oral appliance may have a stimulating electrode(s) positioned on single lateral side of the mouth such as shown in FIGS. 22-23B or on both lateral sides of the mouth. The various extensions described herein with respect to an interoral appliance or a test device can be interchangeable and can be different on each lateral side.

The following examples provide further representative devices and techniques in accordance with the present technology. To the extent any materials incorporated herein by reference conflict with the present disclosure, the present disclosure controls.

### Examples

1. A removeable intraoral electrical stimulation appliance comprising:
an attachment body releasably attachable to a patient's oral cavity and including a lateral segment comprising a posterior molar portion;
a flexible resilient extension comprising an inferior portion and a superior portion; and
an electrode carried by the flexible resilient extension;
wherein the superior portion of the flexible resilient extension is coupled to the posterior molar portion and the inferior portion of the resilient extension is coupled to the electrode; and
wherein the flexible extension is flexible in a posterior-anterior direction.

2. The appliance of example 1 wherein the flexible resilient extension is flexible in a superior-inferior direction.

3. The appliance of any preceding example wherein the flexible resilient extension is flexible in a medial-lateral direction.

4. The appliance of any preceding example wherein the flexible resilient extension permits limited angular movement of the electrode.

5. The appliance of any preceding example wherein the flexible resilient extension permits limited roll.

6. The appliance of any preceding example wherein the flexible resilient extension permits limited pitch.

7. The appliance of any preceding example wherein the flexible resilient extension permits limited yaw.

8. The appliance of any preceding example wherein the flexible resilient extension comprises flexion points.

9. The appliance of any preceding example wherein the flexible resilient extension comprises a spring element.

10. The appliance of any preceding example wherein the flexible resilient extension comprises a point of flexibility that is more flexible than the attachment body.

11. The appliance of any preceding example wherein the flexible resilient extension is more rigid in a medial-lateral direction than in the anterior-posterior direction.

12. The appliance of any preceding example wherein the flexible resilient extension includes a resilient element biased in a medial direction.

13. The appliance of any preceding example wherein the flexible resilient extension includes a resilient element biased in an inferior direction.

14. The appliance of any preceding example wherein the flexible resilient extension includes a resilient element biased in a posterior direction.

15. The appliance of any preceding example wherein the electrode is oriented at an angle between 5 degrees and 90 degrees with respect to a line on a coronal plane, wherein 0 degrees is in the inferior direction.

16. The appliance of any preceding example wherein the electrode is oriented at an angle between 35 degrees and 110 degrees with respect to a line on a sagittal plane, wherein 0 degrees is in the posterior direction.

17. The appliance of any preceding example wherein the flexible extension comprises a posterior arm coupling the electrode to the posterior molar portion, and an anterior arm coupling the electrode to the lateral segment at a location anterior to the posterior arm.

18. The appliance of any preceding example wherein the flexible extension is a first flexible extension, and the anterior arm comprises a first lateral branch and an anterior branch, and wherein the appliance further comprises a second flexible extension laterally opposed to the first lateral extension, the second flexible extension comprising a second anterior arm comprising a lateral branch and an anterior branch wherein the anterior branch of the first anterior arm is coupled to the anterior branch of second anterior arm.

19. The appliance of any preceding example, further comprising a pulse generator coupled to the electrode to provide an electrical pulse to the electrode.

20. The appliance of any preceding example, further comprising a power source coupled to the pulse generator.

21. The appliance of any preceding example, further comprising a sensor configured to sense one or more parameters corresponding to the patient.

22. The appliance of example 21 wherein the one or more parameters comprises a respiration parameter.

23. The appliance of example 21, further comprising a controller programmed with a logic program configured to receive an input from the sensor and to control stimulation in response to the input from the sensor.

24. The appliance of any preceding example wherein the electrode comprises a plurality of electrodes.

25. The appliance of any preceding example wherein at least one of the lateral segment and the flexible resilient extension is customizable for an individual patient's oral cavity.

26. The appliance of any preceding example wherein the electrode is a first electrode and wherein the appliance further comprises a second electrode configured to adhere to an external skin surface.

27. The appliance of example 26, further comprising a first pulse generator coupled to the first electrode and a second pulse generator coupled to the second electrode.

28. The appliance of example 27, further comprising a first power source coupled to the first pulse generator and a second power source coupled to the second pulse generator.

29. A removeable intraoral electrical stimulation appliance comprising:
an anchor device releasably attachable in a patient's oral cavity wherein the anchor device comprises:
   a first lateral segment comprising a first posterior molar portion and a second lateral segment comprising a second posterior molar portion;
   a first stimulating electrode;
   a first flexible resilient extension comprising an inferior portion and a superior portion;
   wherein the superior portion of the first flexible resilient extension is coupled to the first posterior molar portion and the inferior portion of the first resilient extension is coupled to the first stimulating electrode; and
   a second stimulating electrode;
   a second flexible resilient extension comprising an inferior portion and a superior portion;
   wherein the superior portion of the second flexible resilient extension is coupled to the second posterior molar portion and the inferior portion of the second resilient extension is coupled to the second electrode; and
   wherein the first and second flexible extensions are flexible in a posterior-anterior direction.

30. The appliance of example 29 wherein the anchor comprises an anterior portion, and wherein the anterior portion couples the first lateral segment to the second lateral segment.

31. The appliance of example 29 or 30 wherein the flexible resilient extension comprises an anterior arm coupling the first electrode to the anterior portion, and the second flexible connector comprises an anterior strut coupling the second electrode to the anterior portion.

32. A removeable intraoral electrical stimulation appliance comprising:
an attachment body configured to be releasably secured in a patient's oral cavity, the attachment body comprising:
   a first lateral segment, a second lateral segment, and an anterior segment joining the first lateral segment and second lateral segment;
   a first electrode carried by the first lateral segment, and a second electrode carried by the second lateral segment, with the first and second electrodes at least partially facing toward each other; and
   at least one inflatable member carried by the attachment body and positioned to bias at least one of the first and second electrodes into contact with tissue of the patient's oral cavity.

33. The appliance of example 32 wherein the at least one inflatable member includes a first inflatable member positioned to bias the first electrode, and a second inflatable member positioned to bias the second electrode.

34. The appliance of example 33 wherein the first electrode and the first inflatable member are carried by a first flexible extension extending from the first lateral portion, and wherein the second electrode and the second inflatable member are carried by a second flexible extension extending from the second lateral portion.

35. The appliance of example 34 wherein the first electrode is one of two electrodes carried by the first extension, and the second electrode is one of two electrodes carried by the second extension.

36. The appliance of example 33, further comprising a first inflation line coupled to the first inflatable member, and a second inflation line coupled to the second inflatable member, and wherein the first and second inflation lines are independently controllable.

37. The appliance of example 33 wherein the inflatable member is coupled to a pressure sensor to detect at least one of patent movement or patient respiration.

1-37. (canceled)

38. A removeable intraoral appliance, comprising:
an attachment body releasably attachable to a patient's oral cavity; and
an inflatable element carried by the attachment body, the inflatable element being coupled to a sensor to sense a physiologic characteristic of the patient.

39. The intraoral appliance of claim 38, wherein the inflatable element includes a bladder, and wherein the intraoral appliance further comprises:
an inflation line coupled to the bladder and to an inflation medium source to apply an inflation medium to the bladder; and
wherein the sensor is operably coupled to the bladder to sense a change in a pressure of the inflation medium at the bladder.

40. The intraoral appliance of claim 39, wherein the bladder is a first bladder, the inflation line is a first inflation line, and the sensor is at least one sensor, and wherein the intraoral appliance further comprises:
a second bladder;
a second inflation line coupled to the second bladder and to the inflation medium source; and wherein:
the at least one sensor is configured to detect a difference in pressure between the first bladder and the second bladder.

41. The intraoral appliance of claim 40 wherein the at least one sensor includes a first sensor coupled to the first inflation line and a second sensor coupled to the second inflation line.

42. The intraoral appliance of claim 39 wherein the bladder is positioned to undergo a change in internal pressure in response to a change in the patient's respiration characteristics.

43. The intraoral appliance of claim 39 wherein the bladder is positioned to undergo a change in internal pressure in response to a change in the patient's body position.

44. The intraoral appliance of claim 39 wherein the bladder is positioned to undergo a change in internal pressure in response to a change in the patient's tongue position.

45. The intraoral appliance of claim 38 wherein the sensor includes an electrode carried by the inflatable member.

46. The intraoral appliance of claim 38, further comprising a power supply carried by the attachment member and positioned to provide power to stimulation electrodes.

47. A method for sensing physiologic characteristics of a patient via a removable, intraoral appliance, comprising:
inflating at least one inflatable element carried by the intraoral appliance; and
receiving, via a sensor coupled to the at least one inflatable element, a signal corresponding to a change in a physiologic characteristic of the patient.

48. The method of claim 47, wherein the change in a physiologic characteristic includes a change in pressure exerted on the at least one inflatable element by the patient.

49. The method of claim 48 wherein the change in pressure corresponds to a change in the patient's body position.

50. The method of claim 48 wherein the change in pressure corresponds to a change in the patient's tongue position.

51. The method of claim 48 wherein the change in pressure corresponds to a change in the patient's respiration.

52. The method of claim 47 wherein the inflatable element carries an electrode, and wherein the physiologic change is a change in an electrical characteristic of the patient.
